(19) United States
(12) Patent Application Publication
(10) Pub. No.: US 2022/0157143 A1
(43) Pub. Date: May 19, 2022

(54) Baby Vitals Monitor

(71) Applicant: VitalTech Properties, LLC, Plano, TX (US)

(72) Inventors: Anjan Panneer Selvam, Plano, TX (US); Peter Ianace, Frisco, TX (US)

(73) Assignee: VitalTech Properties, LLC, Plano, TX (US)

(21) Appl. No.: 17/441,013

(22) PCT Filed: Mar. 22, 2020
(86) PCT No.: PCT/US20/24100

§ 371 Date: Sep. 20, 2021

Related U.S. Application Data
(60) Provisional application No. 62/890,847, filed on Aug. 23, 2019.
(60) Provisional application No. 62/851,513, filed on May 22, 2019.
(60) Provisional application No. 62/822,274, filed on Mar. 22, 2019.
(60) Provisional application No. 62/822,381, filed on Mar. 22, 2019.
(60) Provisional application No. 62/822,434, filed on Mar. 22, 2019.

(51) Int. Cl. G08B 21/02; A61B 5/11; A61B 5/00

(52) U.S. Cl. CPC G08B 21/0211; G08B 21/0225; G08B 21/0261; G08B 21/0272; G08B 21/0286; A61B 2505/09; A61B 5/112; A61B 5/4803; A61B 5/681; A61B 5/7455; A61B 2503/06; A61B 5/1118

(57) ABSTRACT

A method for monitoring a child utilizing a smart wearable. Biometrics of the child are measured utilizing at least the smart wearable. A status of the child and of the smart wearable is monitored. An alert is generated in response to determining the child has had an impact greater than a threshold, moves beyond a geo-fenced area, one or more of the biometrics exceeds a threshold, and determining the smart wearable has been cut, damaged, or broken.

### PRIORITY

This application claims priority to U.S. Provisional Patent Application Nos. 62/822,274, 62/822,381, and 62/822,434 filed Mar. 22, 2019, U.S. Provisional Application No. 62/851,513 filed May 22, 2019, and U.S. Provisional Application No. 62/890,847 filed on Aug. 23, 2019, hereby incorporated by reference in their entirety.

### BACKGROUND

### I. Field of the Disclosure

The illustrative embodiments relate to biometrics and risk analysis. More specifically, but not exclusively, the illustrative embodiments relate to a system, method, smartwatch, and wearable for monitoring a user's well-being.

### II. Description of the Art

Advances in technology have significantly benefited large portions of the world's population. As a result, individuals have become more accustomed to technology at younger and younger ages. For many very young individuals and those with disabilities, it may be very hard to express oneself beyond the innate ability to cry. It is often difficult to determine why the child or individual is crying and what they may need. In like manner, some technology advancements that may benefit aging users may also be applicable to younger users.

### BRIEF DESCRIPTION OF THE DRAWINGS

Illustrated embodiments are described in detail below with reference to the attached drawing figures, which are incorporated by reference herein, and where:

FIG. 1 illustrates a front view and side views of a smartwatch in accordance with an illustrative embodiment;

FIGS. 2-3 are a partial view of a smartwatch in accordance with an illustrative embodiment;

FIG. 4 is a pictorial representation of users wearing a smartwatch in accordance with an illustrative embodiment;

FIG. 5 is a pictorial representation of a block diagram of a smartwatch 500 in accordance with an illustrative embodiment;

FIG. 6 is a pictorial representation of modules utilized by the smartwatch of FIG. 5 in accordance with an illustrative embodiment;

FIG. 7 is a flowchart of a process for setting up a smartwatch in accordance with an illustrative embodiment;

FIG. 8 is a flowchart of a process for identifying a user in accordance with an illustrative embodiment;

FIG. 9 is a flowchart of a process for sending alerts or notifications in accordance with an illustrative embodiment;

FIG. 10 is a flowchart of a process for generating an alert in response to the smartwatch being removed in accordance with an illustrative embodiment;

FIG. 11 is a flowchart of a process for receiving information from a tag in accordance with an illustrative embodiment;

FIG. 12 is a flowchart of a process for sending an alert for a smartwatch in accordance with an illustrative embodiment;

FIG. 13 is a flowchart of a process for providing stimuli to a child in accordance with an illustrative embodiment;

FIG. 14 is a flowchart of a process for categorizing a cry in accordance with an illustrative embodiment;

FIG. 15 is a flowchart of a process for categorizing a cry of a child in accordance with an illustrative embodiment;

FIG. 16 is a flowchart of a process for providing feedback regarding gait and speech of a child in accordance with an illustrative embodiment; and

FIG. 17 depicts a computing system in accordance with an illustrative embodiment.

### SUMMARY

According to one aspect, a smart wearable, system, and method for monitoring a child utilizing a smart wearable. Biometrics of the child are measured utilizing at least the smart wearable. A status of the child and of the smart wearable is monitored. An alert is generated in response to determining the child has had an impact greater than a threshold, moves beyond a geo-fenced area, one or more of the biometrics exceeds a threshold, and determining the smart wearable has been cut, damaged, or broken.

Another embodiment provides a method for providing information regarding a user. A cray of the user wearing a smartwatch is detected. Characteristics of the care detected by the smartwatch are determined. Information regarding an associated categorization of the cry and potential response to the cry of the user are communicated.

Yet another embodiment provides a method for providing feedback to a user. A gait and speech of a user are measured utilizing a smartwatch worn by the user. Gait and speech analysis of the user are performed. Feedback is provided to the user and/or designated users regarding the gait and speech of the user.

According to one aspect, a smart wearable for monitoring an individual is provided. The smart wearable includes a band for securing the smart wearable to the arm of the individual, a housing attached to the band, the housing having a top, an opposite bottom and side surfaces, a processor disposed within the housing, a display at the top of the housing and operatively connected to the processor, a wireless transceiver disposed within the housing and operatively connected to the processor, an accelerometer disposed within the housing and operatively connected to the processor, a microphone disposed within the housing and operatively connected to the processor, a heart rate sensor for measuring heart rate of the individual, the heart rate sensor operatively connected to the processor and positioned at the bottom of the housing, and an optical sensor operatively connected to the processor, the optical sensor configured for detecting oxygen saturation within blood of the individual when the individual presses a finger against the optical sensor. The smart wearable is configured to generate an alert based on occurrence of an impact greater than a threshold detected using the accelerometer, a location of the smart wearable, removal of the smart wearable from the arm of the individual and a health biometric of the individual that exceeds a threshold. The smart wearable may further include a locking mechanism operatively connected to the band for preventing the individual from removing the smart wearable from the arm of the individual. The locking mechanism may be engaged or disengaged from the arm using a key. The location may be determined using a plurality of beacons in operative communication with the wireless transceiver. The health biometric may include at least one of the heart rate of the individual and the oxygen saturation level within the blood of the individual. The smart wearable may further include an RFID chip disposed within at least one of the band and the housing. The smart wearable may be configured to convey the alert to a remote device using the wireless transceiver. The smart wearable may be is configured to display the alert on the display.

According to another aspect, a method for providing alerts using a wearable monitoring an individual includes the step of providing the wearable, the wearable comprising a band for securing the smart wearable to the arm of the individual, a housing attached to the band, the housing having a top, an opposite bottom and side surfaces, a processor disposed within the housing, a display at the top of the housing and operatively connected to the processor, a wireless transceiver disposed within the housing and operatively connected to the processor, an accelerometer disposed within the housing and operatively connected to the processor, a microphone disposed within the housing and operatively connected to the processor, a heart rate sensor for measuring heart rate of the individual, the heart rate sensor operatively connected to the processor and positioned at the bottom of the housing, an optical sensor operatively connected to the processor, and the optical sensor configured for detecting oxygen saturation within blood of the individual when the individual presses a finger against the optical sensor. The method may further include receiving at the processor accelerometer data from the accelerometer and determining by the processor if the data from the accelerometer characterizes occurrence of an impact greater than an impact threshold. The method may further include determining by the processor if the data from the accelerometer indicates a removal attempt of the smart wearable from the arm of the individual. The method may further include receiving at the processor heart rate sensor data from the heart rate sensor and determining if the heart rate sensor data indicates that heart rate of the individual is beyond a threshold for heart rate. The method may further include receiving at the processor oxygen saturation data from the optical sensor and determining if the oxygen saturation data indicates that blood oxygen level of the individual is beyond a threshold for blood oxygen level. The method may further include generating an alert if (1) the impact is greater than the impact threshold, (2) the heart rate of the individual is beyond the threshold for the heart rate, (3) the blood oxygen level of the individual is beyond the threshold for blood oxygen level, and/or (4) the removal attempt is indicated. The method may further include displaying the alert on the display. The method may further include communicating the alert using the wireless transceiver to a remote device. The wearable may further include an RFID chip and wherein the method further comprises determining a location of the wearable using an RFID interrogator in communication with the RFID chip. The method may further include determining a location of the wearable such as by using the wireless transceiver and at least one beacon in operative communication with the wireless transceiver. The method may further include generating the alert based on the location of the wearable. The wearable may further include a locking mechanism operatively connected to the band for preventing the individual from removing the smart wearable from the arm of the individual. The locking mechanism may be engaged or disengaged from the arm using a key.

According to another aspect, a smart wearable for monitoring an individual is provided. The wearable may include a band for securing the smart wearable to the arm of the individual, a housing attached to the band, the housing having a top, an opposite bottom and side surfaces, a processor disposed within the housing, a display at the top of the housing and operatively connected to the processor, a wireless transceiver disposed within the housing and operatively connected to the processor, an accelerometer disposed within the housing and operatively connected to the processor, a microphone disposed within the housing and operatively connected to the processor, a heart rate sensor for measuring heart rate of the individual, the heart rate sensor operatively connected to the processor and positioned at the bottom of the housing, an optical sensor operatively connected to the processor, the optical sensor configured for detecting oxygen saturation within blood of the individual when the individual presses a finger against the optical sensor, and an RFID chip disposed within at least one of the band and the housing. The smart wearable may be configured to generate an alert based on occurrence of an impact greater than a threshold detected using the accelerometer, removal of the smart wearable from the arm of the individual and a health biometric of the individual that exceeds a threshold. The wearable may further include a locking mechanism operatively connected to the band for preventing the individual from removing the smart wearable from the arm of the individual. The wearable may be further configured to generate the alert based on a location of the smart wearable and wherein the location is determined using a plurality of beacons in operative communication with the wireless transceiver. The health biometric may include at least one of the heart rate of the individual and the oxygen saturation level within the blood of the individual.

According to another aspect, a method for sending alerts from a wearable is provided. The method includes determining baseline readings for a user wearing the wearable, performing sensor measurements to assess a user utilizing optical sensors, gyroscopes, and accelerometers, wherein the smartwatch is worn by a user. The method further includes determining of the sensor measurements compliance with one or more thresholds and sending an alert based on an alert matrix associated with the user. The step of determining the baseline readings may further include establishing a user for the smartwatch, determining the baseline readings including biometrics, behaviors, and patterns for the user, receiving the one or more thresholds for generating an alert or notifications based on the baseline readings and sensor measurements, and generating the alert matrix. The method may further include establishing the user for a wearable, establishing one or more identifiers for the user, and saving the one or more identifiers for future authentication. The method may further include receiving a wireless signal from a tag, determining information associated with the tag from the wireless signal, and providing an indicator associated with the information from the tag. The plurality of tags may be used to establish a geo-fence for the user wearing the wearable. The wireless signals from the plurality of tags indicate a location of the user. Wireless triangulation or signal strength tracking may be performed utilizing the signals from Wi-Fi, cellular (e.g., LTE), or the plurality of tags. The method may further include sending the alert in response to the alert matrix, wherein the alert matrix includes gait analysis, impact or actions threshold, voice command, touch or pinch pattern on the wearable, geo-fence violation, biometric thresholds, and combinations thereof. The method may further include sending the alert in response to an alert matrix.

According to another aspect, a smart wearable for monitoring an individual includes a band securing the smart wearable to the arm of the individual, wherein the band is secured in place, a heart rate sensor measuring the heart rate of the individual, a gyroscope determining an orientation of the individual, an accelerometer detecting impacts of the smart wearable; and a microphone configured to receive sounds from the infant and an environment associated with the individual. The smart wearable may be a smart bracelet worn on the wrist of the individual. The band may be secured utilizing a locking mechanism. The locking mechanism may be engaged or disengaged from the arm of the individual utilizing a key. The key may be a circle key or an electromagnetic key. The smart wearable may be further configured to send an alert in response to determining the infant has had an impact greater than a threshold, left a geo-fenced area, has a health biometric that exceeds a threshold. The location of the smart wearable may be detected by a plurality of beacons. The smart wearable may communicate biometrics of the individual, a status of the individual, and a location of the individual through wireless signals periodically for storage and access in one or more network devices. The alert may be communicated in response to the smart wearable being removed from the individual without authorization. The alert may be communicated in response to the band being cut or removed from the arm of the individual without authorization. The smart wearable May detect cutting, damaging, or breaking the smart wearable to send the alert such as by analysis of data from the accelerometer. The alert may be generated by the smart wearable if it is still operational, and wherein the alert is generated by a network device if the smart wearable is not still operational.

The illustrative embodiments provide a system, method, and smartwatch for determining an impact threshold score. Inputs for height, weight, activity level, hydration information, and age of a user are received. A body mass index of the user is determined. Values for the activity level, body mass index, hydration information, and the age of the user are assigned. The impact threshold score is calculated utilizing the values. Thresholds for a smartwatch are established in response to the impact threshold score.

In other embodiments, the inputs may be received audibly utilizing at least the smartwatch. The inputs may also be received from any number of external devices, systems, equipment, or components. The inputs may be received through the user interface of the smartwatch including at least one or more buttons, a microphone, and a touch screen. The user may be prompted to provide the inputs for height, weight, and age of the user and the activity level and hydration information for the user may be detected utilizing one or more sensors of the smartwatch. One or more optical and conductivity tests may be performed on the user utilizing the smartwatch, the measurements from the test may be compiled, and the measurements may be analyzed to generate the hydration information regarding the user. A menu of options may be presented to the user or a person associated with the user to receive the inputs. The values received from the user may be normalized when assigning the values. A fall prediction assessment may be generated utilizing the thresholds and impact threshold score. Biometrics from the user may be measured utilizing at least the sensors of the smartwatch and an alert may be communicated to the user or one or more authorized parties in response to the biometrics of the user exceeding the thresholds. A hydration mode of the smartwatch may be activated, a determination of whether the user is hydrated may be made, characteristics of the skin of the user may be measured in response to determining the user is hydrated, and a hydration profile for the user may be created utilizing the characteristics for subsequently determining the hydration information. An interior surface and an exterior surface of the smartwatch may include electrodes for determining the hydration information of the user.

Another illustrative embodiment provides a smartwatch for monitoring a user. The smartwatch includes a band securing the smartwatch to the arm of the user. The smartwatch further includes a body of the smartwatch housing logic and at least a portion of one or more sensors the one or more sensors measure biometrics, an activity level, and hydration information of the user. The smartwatch further includes a user interface in communication with the logic configured to receive physiological parameters from the user. The logic determines a body mass index of the user utilizing the physiological parameters, assign values to the activity level, hydration information, and the physiological parameters, calculates an impact threshold score utilizing the values, and establishes thresholds for the smartwatch in response to the impact threshold score.

In other embodiments, the physiological parameters may include at least height, weight, and age of the user. The physiological parameters may be received as input through a user interface of the smartwatch. The interface may include at least a microphone, one or more touch sensors, and one or more buttons. The logic generates one or more alerts for communication through the user interface in response to the biometrics exceeding one or more of the thresholds. The alerts may be sent to one or more authorized devices or users associated with the user. The one or more sensors include one or more optical sensors for measuring the biometrics and conductivity sensors for measuring the hydration information.

Another illustrative embodiment provides a system for monitoring a user. The system includes a server configured to communicate through one or more networks. The server receives inputs for at least height, weight, and age of a user. The system further includes a smartwatch that measures an activity level and hydration for the user utilizing one or more sensors of the smartwatch and communicates the activity level and hydration information from a transceiver of the smartwatch through one or more networks to the server. The server determines a body mass index of the user utilizing the height and weight of the user, assign values for activity level, body mass index, hydration information, and the age of the user, calculates an impact threshold score and thresholds utilizing the values, and communicates the thresholds to the smartwatch for monitoring the biometrics of the user to prevent falls.

In other embodiments, the thresholds may prevent health or medical events associated with the user. The smartwatch may utilize the thresholds to monitor the user and the smartwatch generates one or more alerts for the user and/or authorized users in communication with the user through the transceiver and one or more networks. The smartwatch may utilize optical measurements and/or conductivity measurements from the one or more sensors to determine the activity level, hydration information, and biometrics of the user. The activity level may be determined at least based on motion of the user throughout a time period. The biometrics may be compared against baseline readings for the user. The smartwatch may receive a SIM card for communicating through one or more cellular networks. The smartwatch includes a rechargeable battery.

### DETAILED DESCRIPTION OF THE DISCLOSURE

The illustrative embodiments provide a system, method, device, smartwatch, smart band, or wearable for monitoring a user. The user may represent any number of patients, elderly individuals, persons with disabilities, special needs individuals, regular users, children, or others. As used herein, smartwatch may refer to any device that is worn, adhered to, or positioned on or within the body of the user including bands, sensor modules, anklets, straps, stickers, or other devices. For example, the smartwatch may represent any number of smart wearables that are integrated with or connected to an attachment mechanism, such as a band, strap, sticker, clothing, or so forth. The smartwatch may be worn on the user's wrist, arm, head, neck, leg, chest, shoulder, or so forth.

The smartwatch may be especially beneficial for monitoring at-risk individuals, such as the elderly, sick, or children. These individuals may be particularly susceptible to falls, becoming lost, being abducted/kidnapped, suffering from dangerous medical conditions, or other potentially problematic issues. The smartwatch may be utilized to monitor the user, provide notifications/reminders/alerts, monitor fitness information and biometrics, and send and receive emergency messages.

The smartwatch may be utilized to perform biometric, activity, and location-based monitoring. The smartwatch may provide alerts, notifications, or other indicators to the wearer of the smartwatch as well as any number of other users or devices. For example, emergency alerts may be generated automatically or in response to feedback from the user for protecting the user.

The sensors of the smartwatch including accelerometers, gyroscopes, magnetometers, optical sensors, touch sensors, time-of-flight sensors, blood pressure, blood oxygenation, hydration, mechanical sensors, piezo electric sensors, heart rate, retinal sensors, fingerprint sensors, pressure sensors, microphones, temperature sensors, and water sensors (for one or more of the user, environment, and smartwatch itself). In one embodiment, heart rate, respiration rate, SpO2 (blood oxygenation), ECG, and step count may be valuable vitals captured, analyzed, processed, and communicated by the smartwatch. For example, parameters extracted by an optical sensor (e.g., emitter, receiver, etc.) may include, but are not limited to, heart rate, arrhythmias, heart rate variability (HRV), preventricular contractions (PVC), tachycardia, bradycardia, dehydration, and so forth. The smartwatch may determine calories burned, heart rate variability, perfusion index, activity level, and sleep quality analytics.

The feedback and output devices of the smartwatch may be utilized to provide different stimuli and feedback to the user. In one embodiment, a screen, current/voltage generator, speakers, or a vibration component may be utilized to provide feedback and instructions to the user. As a result, the user may receive information regardless of their capacity to speak or provide verbal or tactile feedback. The smartwatch may provide messages to the smartwatch, such as medication reminders, activity suggestions, user status verifications, and so forth. A touchscreen and large typeface/font may be utilized to provide messages and easy interactions for the user.

The illustrative embodiments are unique in that the smartwatch or wearable may be worn by the user. As a result, the user does not have to hold a device, remember to perform biometric readings, or remember to include an alert device. The various sensors of the smartwatch may work alone and in combination to accurately determine user and environmental information. The smartwatch may score the user's biometrics and status. The smartwatch may be used as a stand-alone device or with other electronic devices (e.g., cell phones, personal computers, etc.) wearables (e.g., hearables, exercise equipment, etc.) or implantable devices. As previously noted, the smartwatch may be worn directly on the user or may be integrated with a shirt, hat, shoe, bag, or other article of clothing or accessory worn or carried by the user.

The smartwatch may generate alerts in response to biometric readings of the user exceeding one or more thresholds. Alerts may also be generated based on the location of the user. For example, a geo-fence may be established around expected locations for the user with alerts generated if the smartwatch is detected outside of those geofenced areas. The user may send an SOS message in response to covering the smartwatch, squeezing one or more portions of the face of the smartwatch or button sequences, tapping the smartwatch in a pattern, or providing other specified input. The command to send an SOS message or calls is as simple as possible to allow an injured, disabled, or otherwise incapacitated user to send the SOS communication. The smartwatch may also perform emergency voice calls utilizing the activation as described.

In one embodiment, the smartwatch may include a locking band. The locking band may be utilized to ensure that the user cannot remove the smartwatch without permission or authorization. For example, the locking band may include a locking mechanism that requires a specific tool to be locked and unlocked. The locking band may also use a digital authorization or signal two lock and unlock the locking band. The locking band may include anti-removal hardware. For example, the band may include wires or conductors that if cut or broken automatically generate an alert. The band may also include a regular latching or securing mechanism that sends an alert when removed from the user. The alert may include sending a message with the last known location and status of the user, generating an audio alert, and sending any number of messages to authorized devices/users.

The smartwatch may also be utilized to perform status, condition, or cry analysis for the user whether child or adult. The smartwatch may determine the biometrics associated with the user's condition to make categorizations and provide information to the user and other authorized individuals. For example, the smartwatch may perform cry measurements and analysis to make categorizations for the user (e.g., hungry, tired, angry, needs diaper change, etc.). The smartwatch may also suggest responses to the user to address the condition of the user. For example, the smartwatch may make suggestions regarding how to care for the user. Information regarding the user may be shared based on permissions, settings, and configuration of the smartwatch.

The smartwatch may also perform gait and speech analysis of the user. The gait and speech analysis may be used to monitor development, growth, rehabilitation, and so forth (e.g., walking, speaking, writing, driving, playing an instrument, etc.). In one embodiment, the smartwatch may be prescribed as a measurement and analysis device or system. The smartwatch may give suggestions, recommendations, or feedback for walking, crawling, wheeling, or other forms of motion.

The illustrative embodiments may be utilized with any number of medical devices (e.g., smartwatches, wearables, implantable, stand-alone, traditional, activity tags, etc.). Extensive information regarding vital signs, emergencies (e.g., fall detection, SOS panic alerts, emergency voice call out, etc.), daily living activities, health, and wellness, and other activities may be recorded. In addition, external databases may be accessed (e.g., environmental, population health, governmental, pollution, alerts, etc.) and updated as needed. The illustrative embodiments may be accessible to the user, caregiver, family, physicians, and others. The illustrative embodiments may provide remote patient monitoring. Automated and user requested messaging may also be performed (e.g., medication reminders, instructions, check-ups, verifications, etc.). The illustrative embodiments may provide a cloud-based, predictive analytics platform to enhance user/patient care.

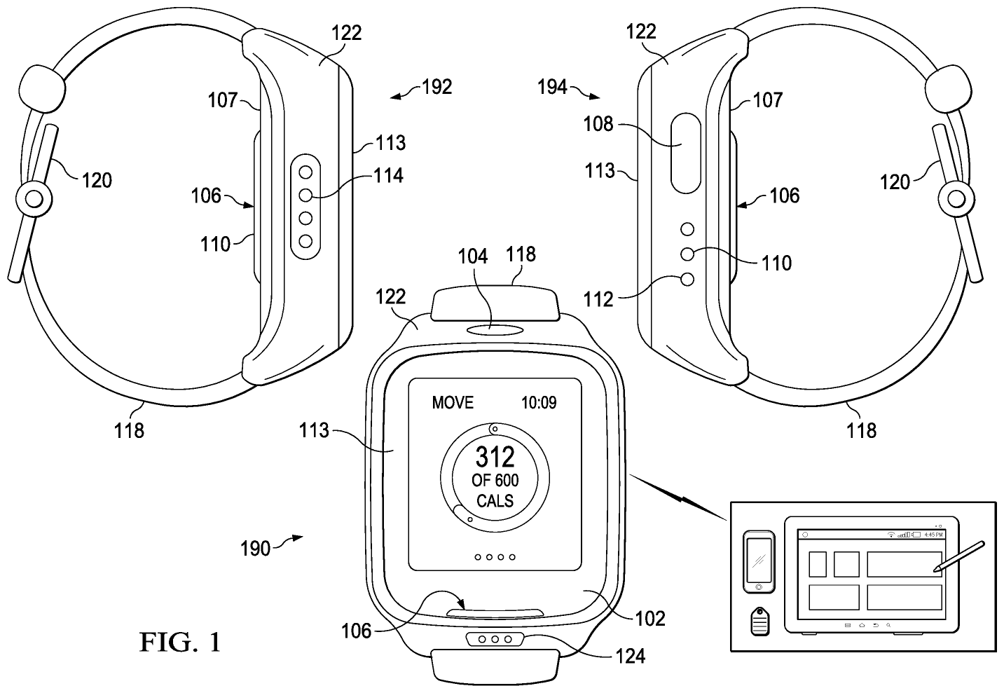

FIG. 1 illustrates a front view 190 and side views 192, 194 of a smartwatch 100 in accordance with an illustrative embodiment. The smartwatch may include a display 102, a camera 104, electrodes 106, button 108, speaker 110, microphone 112, charging pins 114, heart rate sensor 116, a band 118, a latch 120, and a housing 122.

The smartwatch 100 has a housing 122 configured to lie against the user's body and the band 118 configured to hold the housing 122 against the user's body or skin. The housing 122 is shaped and sized to fit on the desired target location for wearing the smartwatch 100, such as on the wrist, ankle, or upper-arm of the user. The housing 122 is a protective case, shell, or platform to which the remaining parts are directly or indirectly attached, integrated, or housed. A plastic or metallic housing structure is expected to be suitable for most embodiments. For example, the housing 122 may be injection-molded plastic, cast magnesium, machined aluminum or steel, a polymer, or other strong materials. The housing 122 may be molded, 3D printed, machined, or otherwise generated. The housing 122 may include seals and other waterproof components. The housing 122 provides a framework that protects the components of the smartwatch 100. The housing 122 may include any number of plastics, biocompatible silicon, polymers (e.g., polycarbonate), glass, stainless steel, and other materials. The sensors may fit flush with the housing 122 or may slightly protrude.

The band 118 may attach to edges of the housing utilizing one or more pins, rods, supports, or so forth. The band 118 may include a support or structure that wraps entirely or partially around the wearer's body to hold smartwatch 100 in place. In the example shown, the band 118 is configured to hold the housing 122 against the wearer's wrist, ankle, or shoulder. The band 118 may also be configured to hold the housing against the head, upper arm, hand, finger, leg, neck, waist, chest, ankle, leg, or additional portion of the user's body. The band 118 may include one or more flexible or rigid straps. The latch 120 The band 118 may also utilize a spring-loaded or interference fit.

In one embodiment, the band 118 may be secured, closed, bound, or joined joinable by a latch 120. The latch 120 may represent a buckle, magnets, buttons, or locking mechanism. The latch 120 may be self-closed or may be secured to the body or clothing of the user. In one embodiment, the latch 120 may lock the band 118 to the body so that it may be only removed utilizing a secured process (e.g., key, digital code, magnetic release, etc.). For example, the latch 120 may be utilized to secure the smartwatch 100 to a user with memory issues, a medical condition, a tendency to wander, an infant or child, or others that may need special care.

In one embodiment, the band 118 may include conductors or other sensors. If cut, broken, or removed without the latch 120 being properly opened, the smartwatch 100 may generate an alert. The smartwatch 100 may also send a running record of the status and location of the user in case the smartwatch 100 is removed, damaged, or purposely destroyed so that the data is kept in a database, cloud system, server, or other device. For example, the status of the user may be sent at a pre-defined interval, such as 30 seconds, 1 minute, 5 minutes, 10 minutes, 30 minutes, 1 hour, or other time period.

The band 118 may be movably or rigidly secured to the housing 122 by pivot pins, a cantilevered anchor, and so on. The band 118 also may be formed integrally with the housing 122. The band 118 may also be removed for securing the body/housing 122 of the smartwatch 100 (without the band 118) within or to clothing, adhesives, third party straps, or so forth. For example, the housing 122 may be directly adhered to the body of the user.

In one embodiment, when configured for use on the wrist, the smartwatch 100 may have the form factor of a traditional watch or fitness tracker. For example, the smartwatch 100 may be formed as a watch, band, or strap. The components of the smartwatch 100 may be integrated into all or portions of the housing 122 and/or band 118. For example, the smartwatch 100 may not include a traditional larger display, but instead may have a smaller display or displays that fits into the narrower band 118. In one embodiment, the band 118 may house flexible electronics. The flexible electronics and circuits may be mounted on a flexible plastic substrate, such as polyimide, PEEK, or transparent conductive polyester film. Various photolithographic techniques may be used to generate the various components and electronics as herein described. In one example, the housing 122 may have a generally flat rectangular or rounded shape (e.g., rectangle, circle, ellipse, etc.) that extends in a plane with a maximum dimension in the plane of approximately two inches or less, and a thickness extending perpendicular to the plane of approximately one quarter inch or less. The band 118 may be rotationally attached to edges of the housing 122 and configured to encircle a volume having a diameter of about two to three inches (e.g., circumference of 6.5-9.5 inches), or such a size as corresponds to the typical dimensions of a human wrist. The housing 122 optionally may be provided with conventional wristwatch features, such as a bezel, face, and mechanical movement or digital clock for telling time.

The smartwatch 100 may also include one or more sensor arrays including a sensor array 106. The sensor array 106 may include one or more sensors configured to collect vital biometric information from the wearer, environmental information, and so on. For example, the sensor array 106 may represent a heart rate sensor. The heart rate sensor may measure the heart rate of the user and the changes or variability of the user's heart rate. One or more portions of the sensor array 106 may be located at an inner surface 107 of the housing 122 (i.e., the surface facing the wearer's body during use). For example, the sensor array 106 may include one or more optical sensors 110 located approximately centrally on inner surface 107 of the housing 122, and oriented to direct one or more spectra of light from the inner surface 107 towards the wearer's body. The optical sensors 110 (or other sensors of the sensor array) may include one or more emitters and receivers. Other components, such as antennas, waveguides, amplifiers, pulse generators, may also be utilized by the sensor array 106. The optical sensors 110 may direct the light, wireless signals, or other emissions at a 90° angle to the inner surface 107, or at an angle less than 90° thereto. Any desired pattern, number, and configuration of optical sensors 110 and associated light sources may be used. Light emitting diodes (LEDs) may be utilized as the optical transceivers 110, but other light sources or emitters may be used in other embodiments. For example, one or more laser sensors may be utilized. The optical sensors 110 may also be referred to as optical transceivers, emitters, and receivers.

The sensor array 106 may include any number pressure, motion, optical, mechano-acoustic, phonocardiograph (PCG), and photoplethysmography sensors. In another embodiment, the inner surface 107 may include one or more radar detectors for detecting blood flow and variability to detect the heart rate of the user. For example, the smartwatch 100 may measure the heart rate of the user using ultra-wideband (UWB) impulse radar. UWB impulse radar may utilize low amounts of power and is harmless to the human body as utilized by the smartwatch 100.

The optical sensors 110 may emit light or wireless signals at one or more wavelengths or spectrum. For example, a first group/components set of one or more of the optical sensors 110 may emit light primarily at about 350-450 nanometers (green light), a second group of one of more of the optical sensors 110 may emit light primarily at about 605-750 nanometers (red light), and a third group of the one or more optical sensors 110 may emit light primarily at about 850-1020 nanometers (infrared light). The wavelengths or signals of each group may be clustered together or distributed among the other groups. The different groups of wavelengths and/or signals may be operated simultaneously or separately, as desired. For example, electromagnetic phase-shift spectroscopy may be utilized as is known in the art. Low-energy electromagnetic waves may be broadcast utilizing any number of transmitters/emitters, transmitters, receivers, transceivers, antennas, and so forth. In one example, the red light and infrared light groups may be alternatively activated to operate in a manner to cause oxyhemoglobin and deoxyhemoglobin in the blood to absorb the different light energies for measurements, and these energy levels may be compared to determine blood oxygen saturation, using techniques known in the art.

The optical sensors 110 are positioned and oriented to both emit and receive back signals reflected from the user's body. The optical sensors 110 may measure light reflected from the user's body to detect the presence, amplitude, phase, attenuation/impedance, and other characteristics of reflected light. Optical sensors, photodiodes, or other light receivers which produce a voltage, current, or signal proportional to the amount of impinging light energy may be utilized. The one or more emitters and receivers of the optical sensors 110 may be positioned in any number of patterns.

The receivers of the optical sensors 110 may be tuned to detect and measure particular wavelengths of light. For example, a first group of optical receivers may have a band-pass filter that only transmits/receives light at a range of about 350-450 nanometers (green light), a second group of optical receivers may have a band-pass filter that only transmits/receives light at a range of about 605-750 nanometers (red light), and a third group of optical receivers may have a band-pass filter that only transmits/receives light at a range of about 850-1020 nanometers (infrared light). As another example, one or more of the receivers may include a multi-band “knife-edge” or narrow band filter that allows light at multiple discrete wavelengths to pass through (e.g., a filter that transmits light at one or more wavelengths within the range of 605-750 nanometers and one or more wavelengths within the range of 850-1020 nanometers). As still another example, the received or reflected signals may be unfiltered.

The sensor array 106 may be located at any suitable location on the inner surface 107 of the housing 122 or the band 118. A location at the geometric middle of the inner surface 107 may provide improved shielding against ambient light, but this is not required as other locations may be utilized. The sensor array 106 also may be located on a protuberance or extensions that extends away from the housing 122 relative to the adjacent portions of the inner surface 117, which may make it more likely that the sensor array 106 will rest firmly against the skin. Such a protuberance or extension may act like a fulcrum that remains in contact with the skin as the housing 122 rocks through a range of motion on the wearer's body. The sensor array 106 may also sit flush with the housing 122 on the interior surface 107. In another embodiment, the sensor array 106 may reside within a receptacle, cavity, or hole within the housing 122 to provide additional protection, enhanced angles for applications of light/signals, or to better eliminate contamination from ambient light.

In one embodiment, the inner surface 117 includes contacts for detecting contact of the smartwatch with the wrist, arm, body, or skin of the user. The contacts may be utilized with other sensors to determine user biometrics, determine the status of the smartwatch 100 (e.g., worn, not worn, charging, etc.) relative to the user, and provide environmental information.

The housing 122 may include any number of sidewalls, exterior surfaces, and structures. The housing 122 may include a unibody structure or joined materials. The housing 122 may also include trim, inserts, or other components that structurally and functionally enable the different components. For example, the different components may include contacts, buttons, switches, touch interfaces, and so forth.

The smartwatch 100 may include one or more user interfaces, such as displays, user inputs, audio speakers, microphones, haptic feedback device (e.g., vibrators or tactile probes), projectors, and so on. The housing may define any number of holes, ports, or outlets for the microphone 112 and speakers 110 to both receive and communicate sound waves. In one example, the outer surface 113 may have a display 102 configured to provide information to the user/wearer or a person assisting the wearer. An exemplary display 102 may include one or more lights, indicators, or displays, such as light emitting diodes (LED), a two-dimensional LED screen, a two-dimensional liquid crystal display (LCD), a touch screen, and soon. The display 102 may be a touch display for providing information and receiving input from the user. For example, the display 102 may be configured to display specific information, such as time of day, biometrics, location information, health/medication reminders, self-care reminders, appointments, and so forth. The display 102 may work in conjunction with the speaker 110, vibrator, lights, contacts, or other user interface components.

An exemplary input may include a button 118, such as a capacitive button, a mechanical button, a momentary switch or the like. The smartwatch 100 may also include dials, switches scroll wheels, or other mechanical components as well as soft-buttons configured to be presented by the display 116 for selection based on the mode, activity, configuration, or user preferences implemented by the smartwatch 100. Multiple displays 116 and multiple buttons 118 may also be used. Functions of the displays 116 and inputs 118 are described in more detail below.

The smartwatch 100 includes charging pins 114. The charging pins 114 may be utilized to charge the smartwatch 100 also may include one or more charging ports, communication ports, or the like. The charging pins 114 may be associated with magnets that may be utilized to connect a charging adapter that may interface with the charging pins 114. The charging pins 114 may also be utilized to perform updates, synchronization, downloads, or other communications between the smartwatch 100 and another device. For example, a charging adapter may include a connector for interface with the charging pins 114 on a first end and a USB connector on another end for connection to a computer, tablet, power adapter/wall outlet, or other power source.

In another example, a mini-USB (universal serial bus) or micro-USB port may be provided on the inner surface 107 or outer surface 113 to selectively connect to a charging and/or communication cable. As another example, a dedicated charging port 120 may be provided on the inner surface 107 or the outer surface 113. The housing 122 also may include one or more charger mounts 122 that are configured to mate with a portable charging device, as discussed in more detail below. The smartwatch 100 may also utilize inductive chargers that utilized one or more magnets or interfaces integrated with the charging pins 114 to properly align the smartwatch 100 with a charging device, cable, cord, or so forth.

It will be appreciated that the various components described as being part of the housing 122 may alternatively be moved to the band 118, or the band 118 and housing 122 may be integrated into a single continuous structure. The components and features of the smartwatch 100 may also be integrated into a band only form factor with integrated smaller displays and so forth.

The smartwatch 100 may include any number of transceivers for communicating with wireless devices 196. The wireless devices 196 may represent any number of smart/cell phones, tablets, computers, beacons, identification tags, network devices, routers, hubs, or so forth. Any number of wireless signals may be utilized, such as Wi-Fi, Bluetooth, cellular signals, Zigbee, and myriad other signals, protocols, standards, and/or variations.

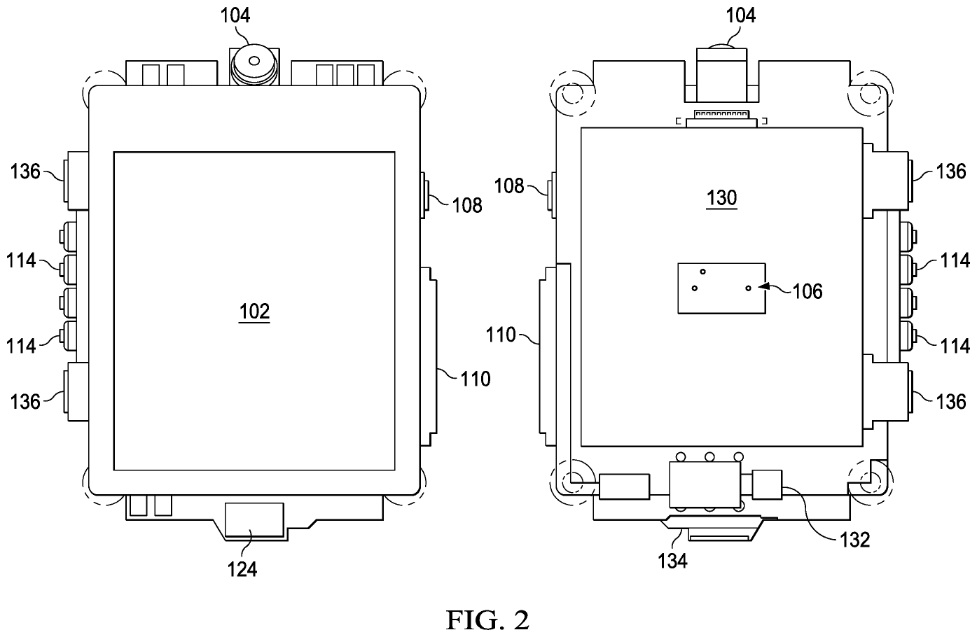

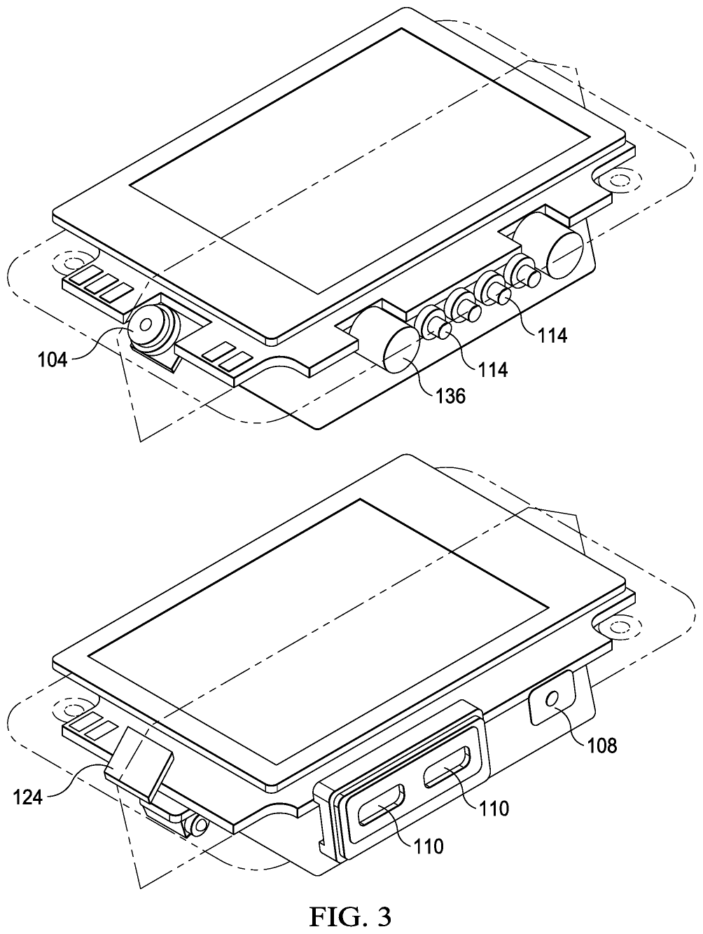

FIGS. 2-3 are a partial view of the smartwatch 100 of FIG. 1 in accordance with an illustrative embodiment. FIGS. 2-3 show a view of the smartwatch 100 with the housing 122 removed or cut-away. The smartwatch 100 may utilize components manufactured by companies, such as Qualcomm, Samsung, Fuji, Taiwan Semiconductor Manufacturing Company (TSMC),

The various components of the smartwatch 100 may be operatively connected to each other and/or one or more processing units (not shown). Any number of traces, busses, wires, fiber optics, cables, wireless interfaces, or other components may be utilized to interconnect the various components of the smartwatch 100. In one embodiment, the display 102 is an AMOLED display with capacitive sensors. The display 102 may also represent liquid crystal displays (LCD), organic light emitting diode (OLED), Micro-LED, or other displays. The display 102 may also be flexible and transparent. Any number of sensors may be integrated into the display 102 itself. The display 102 may be configured to measure information and data (e.g., ambient light, temperature, vibrations, water, humidity, proximity, motion, etc.) regarding the user, environment, or so forth.

The smartwatch 100 may include any number of cameras including the camera 104. The cameras may be integrated into the housing 122, structure of the smartwatch 100, band 118, or housing 122. The camera 104 may be a high-resolution camera varying between 5 megapixels and 20 megapixels or more (pixel size may also vary from 2.0, um, 1.5 um, 1.4 um, 1.12 um, to 0.8 um or less). The camera 104 includes multiple components including optics components and image signal processor (ISP) components. For example, the optical components may include a lens, complementary metal-oxide semiconductor (CMOS) or charge-coupled device (CCD) sensor, infrared filter, and motor control sections. Any number of autofocus modules may be utilized including VCM and MEMs.

The ISP may represent a dedicated digital integrated circuit that processes the image data from the CMOS sensors. The camera 104 may connect to a main board, logic, or other components of the smartwatch 100 through a ribbon cable, bus, or other connector.

The electrodes 106 are conductors through which electricity enters or leaves the smartwatch 100. The smartwatch 100 may include a number of electrodes for performing skin conductivity tests, electrocardiograms, alert generation, and so forth. In one embodiment, the electrodes 106 may be positioned on an interior surface 107 or external surface 109 of the smartwatch 100. For example, an electrode on the interior surface 107 may be conducted through a first arm and the user's second arm/hand may be positioned against one or more electrodes 106 on the exterior surface 109 of the smartwatch. The electrodes 106 may then be utilized to measure the electrical activity produced by the heart as it contracts. The electrodes 106 and logic may check for atrial fibrillation, heartbeat irregularities, and otherwise analyze the electrical activity. The other indicators and interfaces of the smartwatch 100 may also include electrode components for performing the measurements herein described (e.g., display 102, button 108, charging pins 114, heart rate sensor 116, latch 120, etc.).

The button 108 may be a switch or selection component utilized to control a process or function of the smartwatch 100. The display 102 may also display any number of virtual controls for controlling the hardware, software, and functionality of the smartwatch 100. In one embodiment, the button 108 may be a power key for turning the smartwatch 100 on/off. For example, selection of the button 108 may be utilized in conjunction with a confirmation selection received by the display 102 (e.g., touch verification, swipe, etc.). The button 108 may also be utilized to change the operating mode of the smartwatch 100. For example, the smartwatch 100 may include a low-power monitoring mode for user status and well-being, fitness tracking for tracking the activity and actions of the user, diagnosis/testing mode for performing one or more tests, a low power location sharing mode for sharing the location of the user, a sleeping mode, a full power mode, an off mode, and any number of other modes that may be needed or required for the user. In one embodiment, the button 108 may also act as an emergency/SOS button. For example, in response to being pressed/activated for longer than 5 seconds, an emergency alert may be communicated. The user may also be prompted to give a verbal request if possible as part of the emergency message. The button 108 and other buttons, touch sensors, and electrodes may be utilized with different button presses (e.g., short, long, held, combined, push combinations, etc.) to activate the various features of the smartwatch 100.

The smartwatch 100 may also include a number of other buttons integrated with the housing 122 or band 118. Combinations of button selections may be utilized to send a help request alert message or so forth.

The smartwatch 100 may also include external sensor 124. The external sensor 124 may be a pulse oximeter for measuring the blood oxygenation and saturation of the user (SpO₂). In one embodiment, the user may be prompted to place a finger on the external sensor 124 at predefined intervals, in response to user biometrics or as needed to determine the health and wellbeing of the user. For example, the external sensor may utilize an infrared light, photo detectors to transmit light through a translucent, pulsating arterial bed which is a finger in this case. The external sensor 124 may also be utilized on other portions of the body of the user. The external sensor 124 may utilize transmissive and/or reflective signals to make measurements. The external sensor 124 may also include an ambient light detector. The housing 122 may include any number of other external ambient light sensors. In one embodiment, the user may send an emergency alert or request for help by simply covering the smartwatch 100 with their hand for a predetermined amount of time. The smartwatch 100 may vibrate and send messages indicating that the emergency message is going to be sent to prevent unwanted messages from being sent.

The speaker 110 may communicate the generated sounds through one or more ports or openings in the housing 122. Although not shown, the speaker 110 may include components such as digital-to-analog converters, amplifiers, attenuators, filters, and/or other components necessary for the speaker 110 to convert an electrical signal into a sound wave. In one embodiment, the speaker 110 may include multiple speakers including a tweeter, a mid-range, and a bass speaker or associated component. In another embodiment, the speaker 110 may be configured to generate vibration signals or patterns communicated through the user's ear/head or bone structure.

The microphone 112 it is a component for converting soundwaves into an electrical signal which may be amplified, recorded/saved, or communicated. The microphone 112 may receive voice commands from the user or other authorized parties or receive ambient sounds. The measurements performed by the microphone 112 may be utilized in real-time or saved for subsequent analysis, processing, or communications. For example, sounds may be stored in a memory (not shown) to be processed by one or more processors of the smartwatch 100 to determine the ambient noise, location, individuals/animals near the user, and other applicable information. The microphone 112 may include components, such as analog-to-digital converters, amplifiers, attenuators, filters, and/or other components necessary for the microphone 112 to convert a sound wave into an electrical signal. Voice commands received by the microphone 112 may be used by one or more programs or applications executed by the processor for controlling one or more functions of the smartwatch 100. In one embodiment, sounds, speech, and noises detected by the microphone 112 may be utilized to automatically adjust or tune the mode, settings, configuration, sensors, or other components and functions of the smartwatch 100. For example, the user may be more closely monitored in audio conditions known to cause stress to the user (e.g. large groups, traffic, etc.).

As noted, ambient sounds received by microphone 112 may be used by the processor 14 for calibrating or modifying components and functions of the smartwatch 100. For example, the processor may execute an application stored on the memory 20 adjusting the volume of alerts played by the speaker 110 in loud environments, such as “your heart rate is elevated, please sit down in a quiet area for five minutes.”

The charging pins 114 are connectors for charging a battery 130 of the smartwatch 100. The charging pins 114 may be connected to any number of regulators, amplifiers, and other circuits and logic for charging the battery 130. In another embodiment, the charging pins 114 may be replaced by an inductive charger. The inductive charger may allow the batter 130 of the smartwatch 100 to be inductively charged.

The battery 130 may be of any type suitable for powering the smartwatch 100, such as a lithium ion battery. In one embodiment, the smartwatch 100 may be powered by a fuel cell, solar cell, ultra-capacitor, piezo electric generator, thermal generator, or so forth. Alternative battery-less power sources, such as sensors/receivers configured to receive energy from radio waves or other types of electromagnetic radiation, may be used to power the smartwatch 100 in lieu of an energy source, such as the battery 130. In one embodiment, the processor of the smartwatch 100 may shut down components or features of the smartwatch 100 to preserve the battery life. For example, the smartwatch 100 may shut down the transceivers and non-essential sensors to extend the battery life in a lower power or emergency mode.

The smartwatch 100 may also include light emitting diodes or indicators presented on the display 102 indicating the battery charge, estimated battery life remaining, smartwatch status, user status, alerts, and so forth. For example, a blue light may represent a full battery, a green light may represent a high level of battery life, a yellow light may represent an intermediate level of battery life, a red light may represent a limited amount of battery life, and a blinking red light may represent a critical level of battery life requiring immediate recharging. In addition, the battery life may be represented by LEDs as a percentage of battery life remaining or may be represented by an energy bar having one or more LEDs. For example, the number of illuminated LEDs represents the amount of battery life remaining in the smartwatch 100. In one example, a connector may interface with the charging pins 114 to recharge the smartwatch 100 through USB charging. In another embodiment, the charging pins 114 may also be utilized for updates to the

The vibrator 132 is a small motor that is partially off-balanced with respect to a mass distribution attached to the motor's shaft/axis. The irregular weight causes the vibrator 132 to vibrate when activated with a current. For example, the vibrator 132 may represent an eccentric rotating mass vibration motor (ERM), a linear resonant actuator (LRA), or a coin motor. The vibrator 132 may be utilized to provide haptic or tactile feedback to the user.

In one embodiment, the sensor array 106 may represent a heart rate sensor. The heart rate sensor may detect the heart rate of the user, variability, average/median heart rate, and any number of other mathematical or statistical measurements. The heart rate sensor may include the optical sensors, one or more electrical contacts, radar sensors, and other applicable sensors for measuring blood flow/movement, vein/blood expansion and contraction, electrical signals, and other applicable information to measure the heart rate of the user. In one embodiment, a combination of optical and electrical information may be utilized and compared to ensure that the sensor array 106 accurately detects the heart rate of the user. In one embodiment, the optical sensors may utilize photoplethysmogram to measure how much blood the hart is pumping under the surface of the skin. The sensor array 106 may also include pulse oximetry sensors to measure the amount of oxygen in the blood of the user. In one embodiment, the sensory array 106 may work in conjunction with other buttons or components to perform an electrocardiogram (ECG or EKG). The sensory array 106 may also track activity, movements, and steps of the user.

The smartwatch may also include a connector 134. The connector 134 may represent a card connector or port for receiving any number of SIM cards (e.g., mini, micro, nano, virtual, etc.) for GSM, PCS, GPRS, SMS, MMS, and other communications protocols, standards, or signals. The connector 134 may also include a port or be configured to receive one or more SD cards to expand the memory or capabilities of the smartwatch 100. The housing may define an opening or port for adding or removing SIM/SD or other cards to the SIM connector 134. For example, the smartwatch 100 may utilize a nano-SIM. The connector 134 may also receive a card that provides a transceiver on a chip for proprietary signals or additional channels or capacity for Bluetooth, Wi-Fi, Zigbee, Z-wave, near-field communications (NFC), industrial-scientific-medical (ISM), radio frequency identification (RFID), infrared (IR), NFMI, and so forth. The connector 134 may also receive additional systems on chip (SoC), additional processing devices, or so forth.

The magnets 136 may sure a charging adapter in place (e.g., against, in contact with, or proximate the charging pins 114 when charging the battery 130 of the smartwatch 100. For example, the position and polarity of the magnets may correspond for attachment with a charging/power adapter.

The smartwatch 100 may also include antennas 138. The antennas 138 may be configured to communicate any number of signals, protocols, or standards. For example, the antennas 138 may be configured for Wi-Fi, Bluetooth, and cellular communications. Other proprietary or other standards may also be implemented by the smartwatch 100.

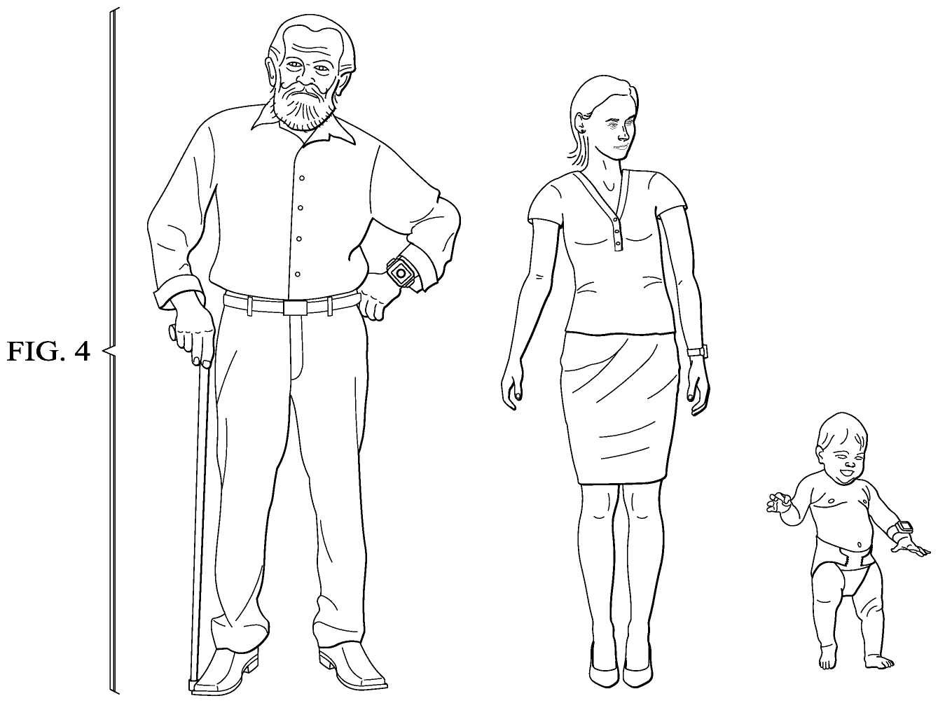

FIG. 4 is a pictorial representation of users wearing a smartwatch in accordance with an illustrative embodiment. Various users 402 may utilize the smartwatch 400 including a toddler 404, an adult 406, and an elderly user 408. The smartwatch 400 may be utilized by all age groups, genders, and persons without limitation. The smartwatch 400 may be particularly useful for monitoring users 402 that may benefit from or require monitoring.

In one embodiment, the toddler 404 may represent an infant, toddler, adolescent, or child (0-18+) that require monitoring for health, behavioral, mental, or other issues, problems, disease, tendencies, or so forth. The smartwatch 400 may be associated with one or more particular locations (e.g., home, school, business, care facility, hospital, etc.). The smartwatch 400 may also allow for the free travel and movement of the users 402.

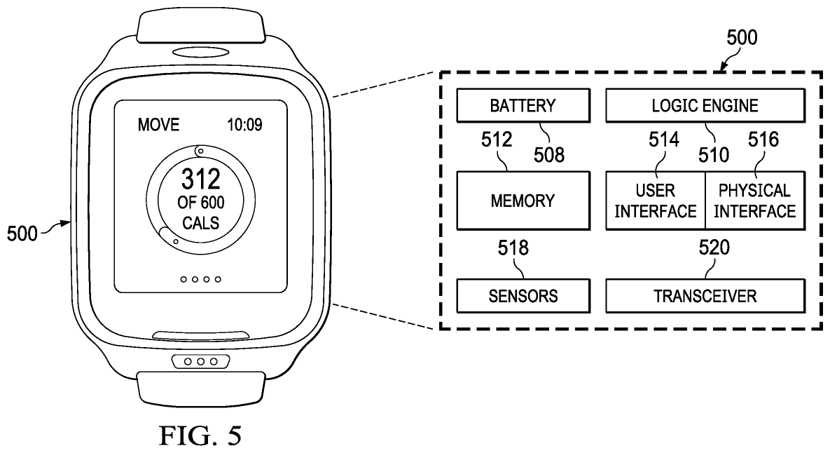

FIG. 5 is a pictorial representation of a block diagram of a smartwatch 500 in accordance with an illustrative embodiment. The smartwatch 500 may include any number of operatively connected components including a battery 508, a logic engine 510, a memory 510, a user interface 514, physical interface 516, sensors 518, and transceivers 520. The smartwatch 500 may have any number of electrical configurations, shapes, and colors and may include various circuitry, connections, and other components. All or portions of the components shown and described with regard to the smartwatch 500 may be included in each of the applicable smartwatches or embodiments thereof. For example, some sensors may be included in various smartwatches 500 for monitoring elderly individuals without inclusion in other embodiments utilized for children. Although not specifically shown, the components of the smartwatch 500 may be connected or communicate utilizing any number of wires, traces, buses, interfaces, pins, ports, connectors, boards, receptacles, chip sets, transceivers, transmitters, receivers, or so forth.

The battery 508 is one or more power storage devices configured to power the smartwatch 500. In other embodiments, the battery 508 may represent a fuel cell, thermal electric generator, piezo electric charger, solar charger, ultra-capacitor, or other existing or developing power storage technologies. The battery 508 may also utilize self-powering features, such as self-winding, solar generation, and thermal energy generation (e.g., body heat).

The battery 508 may be rechargeable or single use. In one example, the battery 508 may be easily inserted or removed regardless of whether it is rechargeable or not. The physical interface 516 may include charging pins or a port for charging the battery 508. The battery 508 and physical interface 516 may include circuitry, wiring, hardware, and electronic logic and control systems to control charging of the battery 508. The charging pins may provide a direct power connection for charging the battery 508. The charging pins may also align an inductive charger with an inductive charging receiver of the smartwatch 500 to charge the battery 508 inductively.

The logic engine 508 is the logic that controls the operation and functionality of the smartwatch 500. The logic engine 508 may include hardware, software, firmware, circuitry, chips, digital and analog logic, or any combination thereof. The digital logic 508 may also include programs, scripts, algorithms, processes, and instructions that may be implemented to operate the smartwatch 500 as well as the components of the smartwatch 500.

In one embodiment, the logic engine 508 is one or more processors. The processor may represent any number of microprocessors, digital signal processors (DSP), application-specific integrated circuits (ASIC), field programmable gate arrays (FPGA), or other applicable devices. The logic engine 508 may utilize information from the user interface 514, is a cool interface 516, sensors 518, and transceiver 522 determine biometric, environmental, and smartwatch 500 status, information, data, and measurements. Any number of inputs, measurements, data, signals, and external data may be utilized by the logic engine 508. For example, the logic engine 508 may make determinations regarding when, where, and how alerts or other communications are sent from the smartwatch 500. The logic engine 508 may both send and receive any number of instructions, commands, or data.

In one embodiment, the logic engine 508 may implement an algorithm allowing the user to associate biometric data as sensed by the sensors 518 with specific commands, user actions, responses, alerts, and so forth. For example, if the smartwatch 500 determines the user has fallen based on feedback from the sensors 518, the user may be asked to verify her physical condition and status to ensure her well-being. A negative response or no response over a designated time period may be utilized to send an emergency communication requesting help, assistance, or a status check. In another example, if the heart rate or blood pressure of the user is above or below high and low thresholds, an audible alert may be played to the user and a communication may be sent through the transceivers 520 to one or more medical professionals, family, friends, or other authorized parties. The logic engine 508 may automatically download and install updates of the operating system, firmware, applications, and so forth. For example, updates may be performed while the device is charging, when the user is sleeping, or at other opportune times.

The memory 512 is a hardware element, device, or recording media configured to store data or instructions for subsequent retrieval or access at a later time. The memory 512 may represent static or dynamic memory. The memory 512 may include a hard disk, random access memory, cache, removable media drive, mass storage, or other construct for storing data, instructions, signals, and/or information. In one embodiment, the logic engine 510 and the memory 512 may be integrated. The memory 512 may represent any type of volatile or non-volatile storage components and processes. The memory 512 may store information related to the status of a user, smartwatch 500, interconnected electronic devices, smartwatch components, and external devices. In one embodiment, the memory 512 may execute and display instructions, programs, drivers, operating systems, or code for controlling the components of the smartwatch 500. The memory 512 may also store the thresholds, conditions, biometric data, user identification information, authorized parties (e.g., utilization, alerts, etc.), geo-fencing information, passwords, available/accessible/authorized devices, user preferences, health records, biometric statistics, parameters, factors, conditions, and so forth.

In some embodiments, linked or interconnected devices may act as a logging tool for receiving information, data, and/or measurements made by the smartwatch 500. For example, a linked wireless device may download data from the smartwatch 500 as available, in real-time, or at designated intervals. As a result, the wireless device (i.e. smart phone, tablet, laptop, home computer, designated hub, etc.) may be utilized to store, display, compile, synchronize, and compile data for the smartwatch 500. For example, the wireless device may display pulse rate, blood oxygenation, blood pressure, blood flow, heart rate variability, temperature, activity, and other applicable biometrics as part of a mobile application executed by the wireless device. The wireless device may also be utilized to receive and display alerts that indicate a specific event or condition has been met. For example, the sensors 518 may detect that the user is experiencing a cardiac event and may notify an associated wireless device to send the message if available. Alternatively, the transceivers 520 may send the alerts to any number of designated users/devices. The smartwatch 500 may offload communications, processing, alerts, and other intensive tasks to an associated communications or computing device to preserve battery power.

The user interface 514 may include any number and type of components for receiving user input and providing information to the user. In one example, the user interface 514 may include a display/visual interface, an audio interface, and a tactile interface. The user interface 514 allows the user to interact with the smartwatch visually, tactilely, verbally/audibly, and through motions. As previously noted, the user interface 514 may include one or more touch displays for displaying and receiving information and selections from the user. The user interface 514 may also include any number of physical buttons, switches, dials, scroll wheels, and other components that may be pushed, activated, turned, moved, pulled, touched, or otherwise activate or deactivate components, features, and functions of the smartwatch 500. Some or all of the buttons of the user interface 514 may have dedicated functions or controls. For example, one of the buttons may be utilized to turn on and off the smartwatch 500 as well as activating an emergency request if held for specified time period (e.g., longer than five seconds). A single button may be configured to perform any number of tasks or functions (e.g., switching between modes, powering on/off the smartwatch 500 or components, entering a different mode, making user selections, sending communications/alerts, activating applications, requesting information, etc.).

As previously noted, the user interface 514 also includes one or more microphones, speakers, vibrator, and electrical contacts. The microphones may receive audio commands, content, and sounds from the user, measure ambient and environmental noises, and otherwise receive any number of audio and sounds. The speakers may communicate audio content including indicators, alerts, verbal audio, information, and so forth to the user. The speakers may also communicate sound waves that may be “felt” by the user rather than heard. The vibrator may also provide tactile feedback and notices as disclosed with regard to the speakers. The electrical contacts may provide a minor electrical current that may also be utilized to communicate with, alert, or check the status of the client. For example, a small current may be utilized to check hydration, heart rate, skin conductivity, provide alerts, determine if the user is conscious, and so forth.

The user interface 514 may utilize any number of screens, windows, or presented information to interface through the touchscreen of the smartwatch 500 or an external device (e.g., laptop, desktop computer, tablet, etc.). The user interface 514 may be customized for the user's needs and condition. Access to vital readings, such as baseline, acceptable, and historic readings, may be quickly accessed and reviewed. The illustrative embodiments secure data and ensure HIPAA compliance and user privacy. Secure messaging may be performed between the user, care teams, medical professionals, guardians, caregivers, and others. In one embodiment, the user interface 514 may provide a single central platform that may be managed by administrators for multiple users. Access to information (e.g., daily vitals, health data, activity data, etc.), secure communications, and alerts may be allowed based on permissions, settings, and other applicable information.

The physical interface 516 may represent any number of components and systems for physically interacting with the smartwatch 500. The buttons of the smartwatch 500 may be integrated with the user interface and/or physical interface 516. The physical interface may include the magnets and charging pins for attaching a power adapter for physical or inductive connection. The physical interface 516 may also include physical interfaces (not shown) for connecting the smartwatch 500 with other electronic devices, components, or systems, such as a charging system, a smart case, or a wireless device. The physical interface 516 may include any number of contacts, pins, arms, or connectors for electrically interfacing with the contacts or other interface components of external devices or other charging or synchronization devices. For example, the physical interface 516 may be a mini or micro USB port. In one embodiment, the physical interface 516 is a magnetic interface that utilizes the charging pins (e.g., pogo pins) to couple to an interface of a power system, the computing device, or the. In another embodiment, the physical interface may include a wireless inductor for charging the wireless earpieces 400 without a physical connection to a charging device.

The sensors 518 may include any number of user or environment sensors. Some of the sensors 518 may be positioned on the interior (worn side) of the smartwatch 500 and others may be externally facing. The sensor may include one or more accelerometers, gyroscopes, magnetometers, optical sensors, blood pressure sensors, radar sensors, chemical sensors, pulse oximeters, ECG/EKG sensors, or other physiological, biological, or environment sensors. Further examples of sensors may include alcohol sensors, glucose sensors, bilirubin sensors, Adenosine Triphospate (ATP) sensors, lactic acid sensors, hemoglobin sensors, and/or hematocrit sensors. For example, a smartwatch 500 for an infant may include a bilirubin sensor for monitoring and treating jaundice. The accelerometers may be utilized to perform wake and motion detection to preserver the battery of the smartwatch 500.

In one embodiment, the sensors 518 may include radar sensors. As described herein, the radar sensors may be positioned to look toward the user wearing the smartwatch 500 or externally from the smartwatch 500. The radar sensors may be configured to perform analysis or may capture information, data, measurements, and readings in the form of reflected signals that may be processed by the logic engine 510. The radar sensors may include Doppler radar, laser/optical radar, or other radar signals, techniques, and processes.

In one embodiment, the sensors 518 may include inertial sensors or other sensors that measure acceleration, angular rates of change, velocity, and so forth. For example, inertial sensors may include an accelerometer, a gyro sensor or gyrometer, a magnetometer, a potentiometer, or other type of inertial sensor. The accelerometer may represent single-axis or multi-axis models. The accelerometer may represent microelectromechanical systems (MEMS) and/or sensors. The accelerometer (or alternatively magnetometer or accelerometer) may detect the position and motion of the user and relative portion of the user's body. The inertial sensors may detect deliberate movements for controlling device functions (e.g., shaking, gestures, turning, etc.).

The sensors 518 include optical sensors. The optical sensors may be utilized to detect user biometrics, ambient light, and so forth. For example, the optical sensor may be configured and utilized as photoplethysmogrpahic (PPG) components that detect blood flow within the user's body. For example, the optical sensor may have an optical emitter that emits light towards the user's skin, and an optical receiver that detects light reflected or absorbed by the blood flowing through the underlying skin and tissue. One or more lasers, LEDs and associated lights sources detectors or components, such as those described above, may be used for this purpose, but other components may also be utilized.

The optical sensors may utilize any number of wavelengths or spectra, such as visible light, infrared (IR), ultraviolet (UV), may be utilized (e.g., X-ray, gamma, millimeter waves, microwaves, radio, etc.). In one embodiment, the spectrometer 442 is adapted to measure environmental wavelengths for analysis and recommendations, and thus, may be located or positioned on or at the external facing side of the wireless earpieces 400.

As the volume of blood in the tissue changes during each heartbeat pulse, the receiver may generate a current or voltage having a waveform corresponding to the change in flow, pressure, volume, or so forth (e.g., PPG data). The heart rate, respiration rate, SpO2, blood pressure, pulse oximetry, and other biometrics may be measured over time. The sampling rate and size of the data sets may vary based on accuracy, resolution, and power utilization that is required. For example, comprehensive data sets may be gathered based on predetermined events, based on the user status, a schedule (e.g., user based, default, set by a medical professional). The sensors 518 may utilize multiple measurements to ensure the accuracy of detected biometric data. In one embodiment, the sensors 518 and logic engine 510 may utilize algorithms to determine biometric data. For example, sample sets may be analyzed in real-time or subsequently. The logic engine 510 may utilize mathematical and statistical processing for the biometrics including Fourier transforms, autoregression, demodulation, and so forth. It will be appreciated that any suitable algorithm may be used to estimate pulse rate, respiration rate, oxygen level, and other biometrics, and various such algorithms.

The smartwatch 500 also may utilize the sensors 518 to discriminate when the smartwatch 500 is being worn or not worn. Proximity sensors and temperature sensors may be used for this purpose. For example, when relying on a proximity sensor to determine whether the device 100 is being worn, a false positive may arise if the device is removed from the person and placed on a surface in contact with the proximity sensor, and the device 100 may continue to operate as if it still being worn.

The sensors 518 may be tuned, biased, offset, calibrated, or otherwise adjusted in real time or after the fact for optimal performance. For example, the sensors 518 may be adjusted for conditions or factors, such as ambient noise, white noise, temperature differentials, performance degradation over time, hardware/software errors, and so forth. The sensors 518 may utilize any number of filters, amplifiers, offsets, or so forth to adjust their respective performance. The sensors 518 may also utilize any number of sampling rates or frequencies. The sampling frequencies may be adjusted based on the activity, user status, designated parameters (e.g., information from medical professionals), and other applicable information. For example, sampling rates may be adjusted up or down automatically or as selected by the user or another authorized party (e.g., person, device, network system, etc.). The sampling rate may be changed to maximize the performance of the sensors 518 or to preserve the battery 508. Any number of mathematical or statistical processes may be utilized to enhance the accuracy and readings made utilizing the sensors 518.

The sensors 518 may also include a global position system (GPS) or global navigation satellite system (GNSS) component/unit to determine the location of the smartwatch 500. Global positioning information may be determined utilizing any number of systems or services (GPS, GLONASS, Beidou, Galileo, etc.). The GPS component may operate continuously, based on events, at predetermined intervals/time periods, or based on other information. One or more thermometers or temperature sensors may measure the environmental temperature as well as the temperature of the user. Galvanic, proximity, or touch sensors may also be utilized to detect the presence of the user (inner surface) as well as exterior persons, objects, animals, and so forth. The sensors 518 may also perform cellular and Wi-Fi positioning (e.g. iZat). In addition, the location of the user may be determined utilizing wireless triangulation (e.g., Wi-Fi, cellular, etc.), beacons, or other similar methods.

Although not specifically shown, the smartwatch 500 may include modular units that may be removed, replaced, exchanged, or otherwise updated. As a result, modular sensor unit may allow the smartwatch 500 and corresponding sensors 518 to be adapted for specific users, purposes, functionality, or needs. For example, the modular sensor unit may have contacts or interfaces for being connected or disconnected. Various applications, such as Android applications, may be installed and executed by the smartwatch 500.

The smartwatch 500 may also include one or more transceivers 520. The transceivers 520 are components including both a transmitter and receiver which may be combined and share common circuitry on a single housing. The transceivers 520 may communicate utilizing Bluetooth, Wi-Fi, LTE, radio frequency identification (RFID), NFMI, ZigBee, Ant+, near field communications, wireless USB, infrared, mobile body area networks, ultra-wideband communications, cellular (e.g., LTE, 3G, 4G, 5G, PCS, GSM, etc.), infrared, or other suitable radio frequency standards, networks, protocols, or communications. The transceivers 520 may also be a hybrid transceiver that supports a number of different communications. The transceivers 520 may also represent independent transmitters and receivers. For example, the transceivers 520 may communicate with other electronic devices or other systems utilizing wired interfaces (e.g., wires, traces, etc.), NFC or Bluetooth communications. For example, the transceivers 520 may allow for induction transmissions with the smartwatch 520. For example, the transceiver 520 may utilize Wi-Fi and long-term evolution (LTE). The transceivers 520 may be utilized for geo location services, two-way voice communications, automated fall detection and messaging (even if the user is unconscious), access control with RFID, and so forth.

The transceiver 520 may represent one, two, three, four or more transceivers that may be separate or share components and circuitry. The transceivers 520 may be utilized to communicate with any number of communications, computing, or network devices, systems, equipment, or components. The transceiver 520 may also include one or more antennas for sending and receiving signals. The transceiver 520 may communicate with any number of networks (e.g., personal area networks, body area networks, etc.) or devices. The smartwatch 500 may include any number of ports for enhancing the logic engine 510, memory 512, sensors 518, or transceivers 520. For example, the smartwatch 500 may receive SIM cards, memory cards, and so forth. The SIM cards may enable any number of communications protocols, standards, or signals. Access to the SIM card may be open or locked for enhanced security. The transceivers 520 may be utilized to update the software or firmware of the smartwatch 500, synchronize data, send alerts, messages, or other communications.

In operation, the logic engine 510 may be configured to convey different information using one or more light emitting diodes or other indicators. The components of the smartwatch 500 may be located on a printed circuitry board(s), chips, circuits, programmable logic, stand-alone components, or a combination of components.

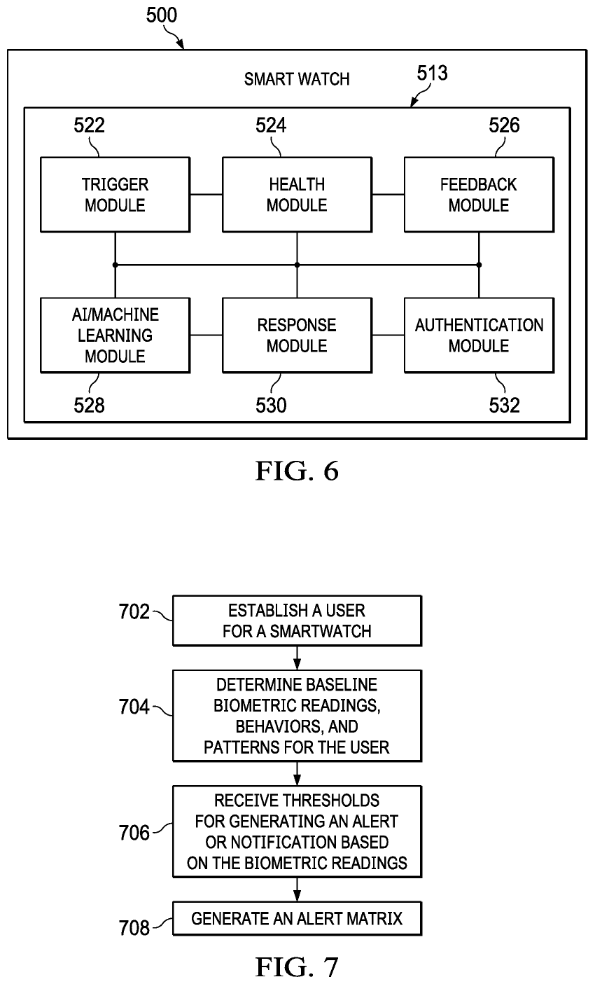

FIG. 6 is a pictorial representation of modules 513 utilized by the smartwatch 500 of FIG. 5 in accordance with an illustrative embodiment. In one embodiment, the modules 513 may be stored in a memory of the smartwatch (i.e., memory 512 of smartwatch 500). The modules 513 may also represent hardware, digital logic, firmware, digital logic, circuits, or a combination thereof. For example, the modules 513 may be hardwired to perform the functions, processes, steps, features, and actions herein described. The modules 513 may be performed for data acquisition, data ingestions, and data warehousing.

The data of the modules 513 may be encrypted, coded, or otherwise secured to prevent unauthorized or unwanted access. Any number of passwords, keys, tokens, identifiers, pins, biometrics, or other data/information may be required to access the modules 513 and their associated data, information, or processes. The user or other authorized agents may specify how, when, and where the information from the modules 513 may be shared with other users/devices. The devices may interact with other Internet-of-thing (IoT) devices or may themselves be IoT devices.

The modules 513 may include a trigger module 522, a health module 524, a feedback module 526, an AI/machine learning module 528, a response module, 530, and an authentication module 532. Any number of other modules may also be uploaded, exchanged, downloaded, programmed, utilized, or implemented. The modules 513 may perform on-user calibration and real-time streaming. The modules 513 may utilize any number of application program interfaces (APIs) to interact with distinct software, devices, and so forth.

The trigger module 522 implement any number of components, actions, features, processes, programs, and other processes (referenced as processes) of the smartwatch 500. The trigger module 522 may include a database, library, or references for any number of thresholds, commands (e.g., verbal, tactile, gestural, etc.), measurements, determinations, or so forth that may be utilized to determine a triggered process. The trigger module 522 may activate, pause, or deactivate any number of processes at any time. As previously noted, the trigger module 522 may be established based on medical preferences provided by default, a medical provider, an institution/facility, parents/children/guardians, or so forth. In one embodiment, the trigger module 522 may be activated based on a heart rate that is above or below a designated threshold (145<HR, 45>HR). The trigger module 522 may include any number of other thresholds for biometrics such as impacts, respiration rate, blood pressure, user noises, motion, or so forth.

The health module 524 manages health information for the user. In one embodiment, user biometrics and health information may be securely stored and accessed utilizing the health module 524. The health module 524 may be utilized to determine the status, condition, and well-being of the user. The health module 524 may indicate the status of a particular condition, disease, malady, abnormality, or other issue. For example, the health module 524 may determine the user's stability associated with vertigo.

The feedback module 526 provides input and output for the smartwatch 500. In one embodiment, the feedback module 526 may provide information to the user. For example, the feedback module 526 may provide instructions, commands, or feedback for determining the well-being of the user. For example, the feedback module 526 may provide instructions for the user to “stand and walk around for five minutes”, “take deep breaths for one minute”, reposition the user's body for better circulation, perform a status test (e.g., balance, vertigo, stamina, endurance, etc.), and perform any number of other processes, steps, or actions. The feedback module 526 may utilize any of the components or systems of the smartwatch 500 to provide feedback to the user, associated/linked devices, third-party users or so forth. The speakers, contacts, vibrator, screen/display/lights, or other components of the smartwatch 500 may be utilized to provide feedback. For example, the feedback module 526 may provide a verbal indicator that the user “needs to be rolled to prevent bed sores” for a patient that is unable to speak or easily communicate.

The AI/machine learning module 528 (hereinafter “learning module 528”) adapts the smartwatch 500 to the needs and requirements of the user or responsible person(s), institution(s), facilities, agents, medical professional(s), or so forth. In one embodiment, the learning module 528 may communicate health information to one or more cloud networks or systems. The health information may be analyzed and processed to provide suggestions, actions, activities, and other information applicable to the user. For example, the learning module 528 may determine baselines, scores, parameters, criteria, and thresholds that may be utilized by the smartwatch 500. The smartwatch 500 may adjust the applicable information in real-time, during scheduled updates, based on age, life events, status, condition, diagnosis, and other applicable information. The machine learning module 528 may utilize deep learning including any number of neural networks. The machine learning module 528 may include a clustering and learning algorithm.

The response module 530 controls how and when communications and messages are sent. The communications may be communicated to the user, a medical professional, family/friends/guardians, third-parties, monitoring services, institutions, facilities, or so forth.

The authentication module 512 identifies or authenticates one or more users that may wear the smartwatch 500. In one embodiment, the smartwatch 500 may be worn by multiple users. For example, the smartwatch 500 may be utilized in a care facility to monitor users. As a result, the smartwatch 500 may be transitioned between different users on any given day (or time period). The authentication module 512 may utilize biometric information to authenticate the user. The authentication module 512 may also utilize other applicable information, such as location, activities, actions, behaviors or other applicable details to identify the user.

FIG. 7 is a flowchart of a process for setting up a smartwatch in accordance with an illustrative embodiment. The process of FIGS. 7-12 may be performed utilizing a smartwatch as herein described. The steps and process of each of the Figures and the corresponding description may be interchangeable, combined, and otherwise utilized in innumerable combinations. The process may begin by establishing a user for a smartwatch (step 702). One or more users may be associated with a single smartwatch. For example, the smartwatch may be utilized as a resource that is placed on users (e.g., residence, patients, students, etc.) that require additional monitoring. During step 702, a user profile may also be established. In one embodiment, questions may be posed to the user utilizing a display, speaker, or other interface components of the smartwatch. Answers, feedback, or input may be received utilizing a touch display, hard or soft buttons, dials, microphones, or other interface components of the smartwatch. In another embodiment, the user profile may be received from a separate device, such as a personal computer, wireless device (e.g., smart phone, tablet, etc.), server, or other device.

Next, the smartwatch determines baseline biometric readings, behaviors, and patterns for the user (step 704). The smartwatch may monitor the actions and behaviors of the user for an hour, day, week, or other time period to determine the baseline/default readings applicable to the user. Steps 704 may allow the smartwatch to determine different information, criteria, parameters, and details associated with the user, such as daily schedule, normal/expected behaviors, activity levels, activities, biometrics (e.g., electro conductivity of the user's skin, average heart rate, heart rate range during physical/stressful activities, etc.). In one embodiment, the smartwatch may ask the user to identify activities, actions, status/frame of mind, and other applicable information during the process of step 704. For example, the user may be presented with a drop down menu of activities (e.g., sleeping, eating, bathing/showering, sitting, walking, chores/cleaning, sexual activity, driving, etc.) or actions, status information (e.g., happy, sad, mad, frustrated, confused, sick, stiff, feeling good, nauseous, sore, lightheaded, etc.), and other applicable selections.

Next, the smartwatch receives thresholds for generating an alert or notification based on the biometric readings (step 706). In one embodiment, the thresholds may be stored internally within the memory of the smartwatch. For example, the thresholds may be specified based on the information from step 704 including the age, sex, height, weight, race, medical conditions, and so forth. In another embodiment, the thresholds may be received from an external source, such as a medical professional or server, through a network connection or other wireless signal. For example, a medical professional (e.g., doctor, nurse, physician's assistant, etc.) may specify the threshold levels for generating alerts or notifications. During step 706, the smartwatch may also receive information regarding how the alerts and notifications are sent (e.g., texts, emails, in-app messages, etc.) including email addresses, user names, phone numbers, IP addresses, passwords, authentication information and details, and other applicable information.

Next, the smartwatch generates an alert matrix (step 708). The alert matrix includes all of the applicable information, details, and data from steps 702, 704, and 706. The alert matrix is utilized to determine how and when the alerts and notifications are sent. The information and data from the flowchart of FIG. 7 may be compiled and utilized across devices. In one embodiment, the alert matrixes from numerous devices may be compiled by a cloud system to perform machine learning and apply logic to suggesting best practices for the alert matrix of other devices.

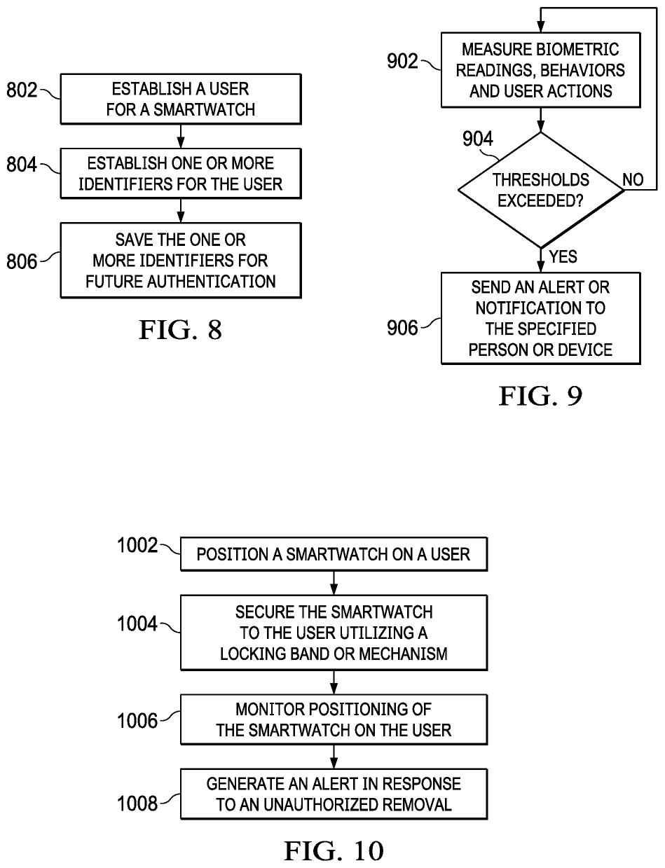

FIG. 8 is a flowchart of a process for identifying a user in accordance with an illustrative embodiment. The process may begin by establishing a user for a smartwatch (step 802). The user may provide applicable information for a user profile, settings, parameters, or other information, such as name, age, address, phone numbers, email addresses, sex, height, weight, race, Internet/wireless service providers, network information, smart home information, medical conditions/issues, allergies, associated medical professionals, associated family/guardians, and other applicable information. The user may also provide additional information and give permissions for the data and information submitted as well as gathered biometrics to be shared. For example, the user may share emergency medical records with the smartwatch or systems associated with the smartwatch (e.g., cloud system, assign servers, etc.).

Next, the smartwatch establishes one or more identifiers for the user (step 804). The identifiers may be automatically read by the smartwatch or may require specific user actions to receive the identifiers. The smartwatch may utilize identifiers or biometrics, such as user names, avatars, pictures, passwords/pins, fingers prints, voice identifiers, ice/iris scans, skin conductivity, location, available wireless devices/signals/networks, height, information from the party devices/users, and other applicable information.

Next, the smartwatch saves the one or more identifiers for future authentication (step 806). The one or more identifiers may be saved within a memory or other storage of the smartwatch. The one or more identifiers may also be communicated to an external device, network, server, database, or system. For example, the identifiers may be saved in an encrypted format that may only be unlocked utilizing a key managed by the user from the smartwatch or other applicable device.

FIG. 9 is a flowchart of a process for sending alerts or notifications in accordance with an illustrative embodiment. The process of FIG. 9 may begin by measuring biometric readings, behaviors and user actions (step 902). The biometrics may be measured utilizing any number of sensors of the smartwatch. In addition, other systems, equipment, components, or devices, such as smart clothing/footwear, smart phones, security systems/camera systems, wearable sensors, smart glasses/jewelry, and implantables may provide relevant information. For example, the smartwatch may receive biometrics or other data and information regarding the user or the environment of the user through any number of wireless communications. Any number of vision or sensor systems may provide relevant information regarding the user/wearer.

Next, the smartwatch determines whether any thresholds are exceeded (step 904). The thresholds may be specified and set utilizing any number of processes, such as those shown in step 706 of FIG. 7. The thresholds may be set by the user himself/herself, parents/guardians/caregivers, medical professionals, or other authorized parties or individuals. If the thresholds are not exceeded during step, 904, the smartwatch returns to measure biometric readings, behaviors, and user actions (step 902).

In response to determining the threshold is exceeded during step 904, the smartwatch sends an alert or notification to the specified person or device (step 906). The alert or notification may be sent in text, audio (e.g., spoken, sound, alarm, etc.), in-application message, email, tactile, or other formats. The alert or notification may be communicated to the user as well or may be withheld to keep the user from panicking or worrying excessively which may make the detected situation worse.

FIG. 10 is a flowchart of a process for generating an alert in response to the smartwatch being removed in accordance with an illustrative embodiment. The process may begin by positioning a smartwatch on a user (step 1002). The smartwatch may be worn on the wrist of the user. The smartwatch may alternatively or optionally be worn on the ankle, arm, leg, neck, or other portion of the user's body. The smartwatch may be positioned to read biometrics and based on the type of users and monitoring being performed.

Next, the smartwatch is secured to the user utilizing a locking band or mechanism (step 1004). In one embodiment, the locking band includes a buckle, latch, or other securing mechanism that may be locked or otherwise activated to detect when removed. For example, the buckle may be locked with a key (e.g., cylinder key, tubular key, RFID lock/key, electromagnetic lock, etc.). In one embodiment, a current may be communicated through the band and buckle. Inadvertent or intentional removal of the smartwatch may be noted. The smartwatch may be utilized on elderly, mentally incapacitated, incarcerated, or young user's that may need additional monitoring. The smartwatch may also be utilized for any number of users that may require special monitoring for health, security, well-being, or other reasons. The smartwatch may be able to detect if the smartwatch is cut, broken off at hinges, buckles, or connectors, or otherwise removed or deactivated. Locations, biometrics, user activities, environmental data, and other information of the smartwatch may be periodically uploaded to a secured database to provide safety information regarding the user. This information may be shared as needed (see step 1008).

In one embodiment, the smartwatch may include a buckle or contacts that indicate that the smartwatch is being worn. For example, an electrical current may be communicated through the buckle or contacts at all times or periodically to ensure that the smartwatch is being worn. An accelerometer and heart rate monitor may also be utilized to detect whether the smartwatch is being worn. For example, based on motion, the smartwatch may ensure that the smartwatch is positioned on the user. A band of the smartwatch may also include wires, contacts, touch sensors, or other components for measuring that the smartwatch is being worn.

Next, the smartwatch monitors positioning on the user (step 1006). The smartwatch may ensure that it is properly positioned along with the corresponding sensors to perform accurate and effective measurements. The smartwatch may provide alerts or notifications if the smartwatch is not properly positioned or worn by the user. The alerts or notifications may be communicated to the user utilizing audio, tactile, text, or externally communicated alerts. For example, the smartwatch may play a message through a speaker to “adjust the smartwatch for sensor readings.” For users that are unable to perform such adjustments themselves, the alerts or notifications may be communicated externally as previously noted. The smartwatch may also provide exact take instructions for adjusting the fit and positioning of the smartwatch. In one example, the smartwatch may have been improperly secured during step 1004. As a result, the user may be provided with specific instructions for adjusting the fit of the smartwatch. The user may also be provided with feedback for cleaning or maintaining the sensors of the smartwatch.

Next, the smartwatch generates an alert in response to an unauthorized removal (step 1008). The alert may be generated for both the user as well as authorized third parties/devices. As previously noted, the alert may include information regarding the user's current or last known location, status, activity, behavior, and so forth. For example, the information regarding the user, smartwatch, and environment may be stored in a smart phone, server, cloud system/network, or other device, system, equipment, or component. As a result, the information may be shared even if the smartwatch is broken, non-functional, or other ways incapacitated. The alert may be sent for the safety of the user. For example, the alert may indicate a health/status event, abduction, accident, or other event has occurred.

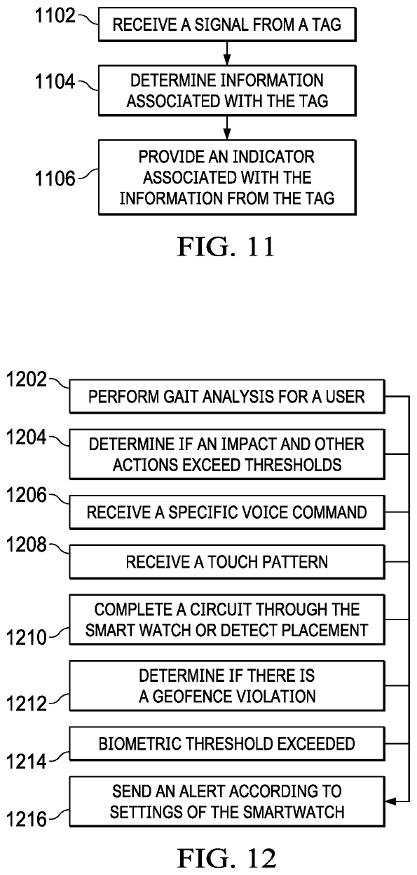

FIG. 11 is a flowchart of a process for receiving information from a tag in accordance with an illustrative embodiment. The tag may represent any number of active or passive devices, such as beacons, radio frequency identification devices, wireless devices, smart clothing, and other devices or components. In one embodiment, the tags may represent communication and location devices that are distributed throughout a location, such as a home, residence, medical facility, nursing location, or so forth. The tags may be particularly distributed at important areas or locations, such as doors, windows, entryways, stairs, furniture (e.g., chairs, beds, couches, exercise equipment, etc.), and so forth. The tags may also represent already existing devices or components, such as computing devices, communications devices, appliances (e.g., microwave, stove, refrigerator, etc.), medical devices (e.g., oxygen generators, heart rate monitors, CPAP machines, etc.), wall outlets, network devices, and so forth.

The process may begin by receiving a signal from a tag (step 1102). The tag may communicate with the smartwatch utilizing any number of wireless signals, protocols, or standards. The tag may be received based on any number of factors, criteria, parameters, events, settings, profiles, or so forth. For example, the tag may communicate with the smartwatch in response to the smartwatch being within a distance threshold of the tag. The signal may also be received in response to an initiating event as determined by the smartwatch, such as a fall/striking event, user biometrics exceeding one or more thresholds, and so forth.

Next, the smartwatch determines information associated with the tag (step 1104). The tag may specify information relating to itself, the environment, user actions/activities, the user, or other specified information. For example, the tag may include location information relevant to a residence of the user. Location information may specify details, such as floor number/name (e.g., first floor, second floor, upstairs, downstairs, basement, etc.), latitude and longitude, physical address, custom location information within the space, and so forth. The tag may also be associated with rooms, activities, actions, or other applicable information. For example, the tag associated with the dining room may have a corresponding name. Likewise, a tag for the bathroom may have a corresponding name, location, and identifying information. The tags may be utilized to determine location and activities of the user as well as any potential problems (e.g., fall in the bathtub, problems getting into the car in the garage, falling asleep on the couch instead of the bed, etc.). The smartwatch or the tags themselves made log or otherwise record the time proximate one or more of the tags or provide relevant information. For example, the information may include proximity of the smartwatch to any number of tags to determine standard daily activities for a profile or baseline readings.

Next, the smartwatch provides an indicator associated with the information from the tag (step 1106). The indicator may be provided to the smartwatch or associated devices. In one embodiment, the smartwatch may be utilized to monitor the activity, health, and well-being of the user based on interaction with the tag. The tag may be one of a network, array, or group of tags. The tags may communicate with each other (e.g., ZigBee, Bluetooth, Wi-Fi, mesh network, etc.) or may act as stand-alone devices. The indicator may provide information for logging the location and activities of the user. This may be useful for when biometric sensors alone are unable to determine where the user is or what she is doing. For example, the tags may act as location beacons that indicate the location, speed, position, orientation, and/or activity/actions of the user. The information captured by the sensors of the tags (e.g., motion, speed, optics/camera (e.g., visible light, infrared, various spectra), proximity, accelerometer, gyroscope, magnetometer, etc.).

The indicator may also provide information associated with the user. The information may provide audio, visual, and tactile information regarding the location (or an item). For example, the indicator may provide details, such as “this is the office”, “you are in the kitchen near the refrigerator”, “you are at the desk”, or other applicable information and details for education, rehabilitation, and so forth.

In one embodiment, the tags may be utilized to create a location-based boundary or geofence. Movements of the smartwatch within the boundaries may be tracked very exactly (e.g., margin of error less than 5 cm). For example, the tags may be location-based beacons that are hardwired into the electrical systems of a residential care facility for tracking a number of residents utilizing smartwatches and/or other devices (e.g., smart phones, smart clothing, tags, etc.). The tags may also be placed outside the geofenced area to further track the location of the user (e.g., in a car, gym, doctor's office, business office, etc.).

FIG. 12 is a flowchart of a process for sending an alert for a smartwatch in accordance with an illustrative embodiment. The process of FIG. 12 may be performed by the smartwatch. As previously disclosed the smartwatch may be part of a system including one or more wireless devices, tags, personal computing devices, personal communications devices, or so forth. In one embodiment, the process and steps of FIG. 12 may represent an alert matrix utilized by one or more devices.

Steps 1202-1214 of FIG. 12 may be performed or determined independently, concurrently, or simultaneously. In one embodiment, multiple steps (e.g., 1202-1214) may be required to perform step 1216. In one embodiment, the smartwatch performs gain analysis for a user (step 1202). The gait analysis may be utilized for any number of purposes including identifying the user and determining the status and condition of the user (e.g., heart attack, stroke, diabetic event, drunk, disoriented, suffering vertigo, etc.). The gait analysis may also be utilized for toddlers, disabled individuals, elderly users, or others to determine progress or maintenance of walking goals. For example, the gait analysis may determine the user's stability, body orientation (e.g., standing straight up, leaning, limping, etc.), gait length, stamina, and so forth.

The smartwatch may also determine if an impact or other actions exceed thresholds (step 1204). During step 1204, the smartwatch may determine whether the user has fallen based on accelerometer data, gyroscope readings, and so forth. The smartwatch may also detect loud noises (e.g., crashes, breaks, slams, etc.) that may be indicative of a dangerous or unwanted event. The presence of water at unexpected times might also note that there is an issue.

The smartwatch may also receive a specific voice command (step 1206). The voice command may be a command, question, or cry for help. The smartwatch may always be listening for commands or may listen for commands in response to one or more of the readings or determinations of steps 1202-1204, 1208-1214. For example, the smartwatch may listen for a voice command in response to an impact exceeding a threshold in step 1204. The voice command may also be noises associated with an injury or incapacitated user, such as moaning, crying, screaming, complete silence, or so forth. In one embodiment, the smartwatch may record audio and/or video of the user along with sensor readings to communicate as part of an alert.

The smartwatch may also receive a touch pattern (step 1208). The touch pattern may represent input of a numeric pin, password, swiping pattern, or combined activation of the one or more interface components of the smartwatch (e.g., touch selections, button presses, dial operation, etc.). The touch pattern may also represent a specific fingerprint or body part activation or scan (e.g., nose print, iris scan, facial recognition). For example, step 1208 may include one or more captured images of the user. For example, in response to one or more of steps 1202-1214, the smartwatch may utilize one or more integrated cameras or external cameras (e.g., home security/wellness, communications pads, laptops, etc.) to capture images of the user and associated environment to be included as part of the alert.

The smartwatch may also complete a circuit through the smartwatch or detect placement (step 1210). The smartwatch may also set a “wear” mode that indicates that the smartwatch is being worn and a “off” mode when the smartwatch is not being worn. A removal alert may be generated for removal or breaking of the smartwatch. In one embodiment, the different portions of the smartwatch may create a circuit that indicates when the smartwatch has been removed. The smartwatch may also utilize the integrated sensors (e.g., optical, infrared, touch, etc.) to determine whether the smartwatch is being worn or positioned on the body of the user (e.g., wrist, ankle, arm, leg, neck, etc.). The smartwatch may also determine whether a latch or locking mechanism is secured and whether the components (e.g., buckle, case, strap, case back, spring bar/pins/hinges, etc.) are interconnected and properly placed on the user. In one embodiment, the case or body of the smartwatch and the band may be integrated. As a result, the smartwatch may be able to determine if it is being worn in response to a latch, buckle, clasp, or other securing or locking mechanism being connected with the presence of the user's body being detected through one or more of the sensors.

The smartwatch may also determine if there is a geofence violation (step 1212). The geofence may represent an electronic fence or boundary associated with the user. The geofence may also represent a virtual boundary. For example, global positioning coordinates may be utilized to specify one or more boundaries for the user (e.g., residence, physical therapy, user's home, gym, etc.). An alert may be sent during step 1216 in response to the geofence being violated.

The smartwatch may also determine if a biometric threshold is exceeded (step 1214). The thresholds may relate to any number of biometrics or combination of biometrics as are outlined herein.

Next, the smartwatch sends an alert according to settings of the smartwatch (step 1216). The alert may be communicated to one or more parties or devices. The parties or devices may be specified by settings, a user profile, medical records, emergency planning files/procedures, or other applicable information. During step 1216, one or more notifications may also be communicated to the user wearing the watch. The notification may act as a warning or alert. For example, based on steps 1202-1214, the user may be encouraged to put the smartwatch back on, re-enter a designated geofence or perimeter, calm down, lie down, take medications, verify their status, perform one or more responsiveness tests, or provide other information, details, commands, or data. The alerts of step 1216 may be sent to monitor and alert users to information regarding the status and well-being of the user of the smartwatch.

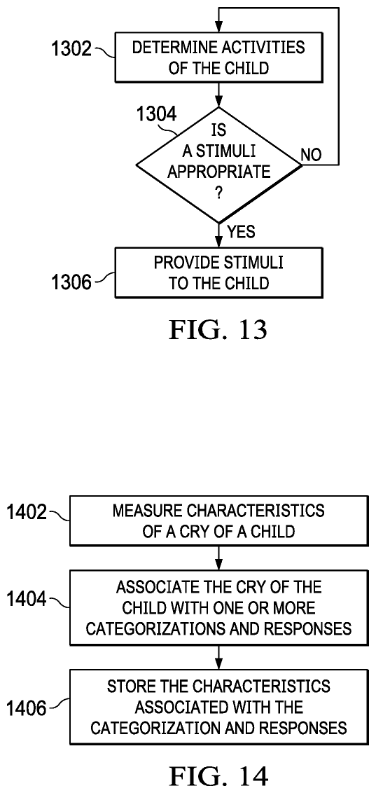

FIG. 13 is a flowchart of a process for providing stimuli to a child in accordance with an illustrative embodiment. The various processes including FIGS. 13-18 may be performed for children from 0-18+. The process of FIG. 13 may also be performed for adults, seniors, or other users with diseases, disabilities, or challenges. The process of FIG. 13 may begin with the smartwatch determining activities of the child (step 1302). The activities of the child may include any number of activities or actions. For example, the activities may include sleeping, eating, walking, sitting, riding a bicycle, climbing stairs, playing, or so forth. The activities may also correspond with the status, mood, or condition of the child, such as laughing, crying, happy, sad, mad, quiet, and so forth. The smartwatch may also determine the time of day, location, proximity to other users/devices/beacons, biometrics, and other applicable information. For example, the smartwatch may be able to determine that the child is crying and upset at day care based on biometrics, time of day, and/or location information.

Next, the smartwatch determines whether stimuli are appropriate (step 1304). The determination of step 1304 may be based on a user profile, thresholds, settings, criteria, parameters, factors, rules, or other information. For example, a parent may have set conditions under which stimuli may be provided to the child wearing the smartwatch, such as extending crying, stressful situations, trigger locations/situations/environments, or so forth. The parent may use an application of a smart phone to specify the rules and conditions for determining when stimuli are appropriate. The information regarding the rules and conditions may then be communicated. In one embodiment, the determination of step 1304 is performed automatically in response to the activities of the child, biometrics, status/condition, parameters, rules, and so forth.

Next, the smartwatch provides stimuli to the child (step 1306). The stimuli may represent one or more actions performed by the smartwatch or devices in communication with the smartwatch (directly or indirectly). The stimuli may represent visual, audio, tactile, electrical, or other stimuli. For example, the stimuli may represent video, music, games, or other stimuli. The stimuli may also represent any number of diversions, distractions, activities, notifications, warnings, messages, or so forth.

In one embodiment, the stimuli may be music or audio content played to the user through the smartwatch. For example, the music may be a favorite song or calming music. Alternatively, the audio may be verbal instructions or content to assist, help, reassure, teach, calm, or guide the user through a situation, event, or circumstance. Similarly, entertaining, educational, or instructional video or images may also be communicated through the smartwatch or other user associated device. The smartwatch may also act as a remote control to one or more speakers, displays, smart televisions, projectors, audio/visual systems, smart phones, tablets, computers, or other devices to play the audio, video or other content.

In one embodiment, the stimuli may be provide information regarding an object, item, condition or so forth. Beacons, tags, transceivers, wireless signals, or other indicators may be utilized to indicate that a stimulus is appropriate in step 1304. Information regarding the object, item, or condition may be provided through the smartwatch. For example, a radio frequency identification tag may indicate the user is near a hot stove and the smartwatch may provide a warning, such as “Jimmy—don't go near the stove.” The stimuli may also indicate the identification regarding an object, such as the name and how to spell and say the object, such as audio form the speaker of the smart watch that indicates “that is a recliner spelled r-e-c-l-i-n-e-r” while the display of the smartwatch may display similar information and may even show a picture of the recliner.

The stimuli may be an education, informational, or warning message to the user. As a result, the smartwatch may provide information that facilitates the learning, protection, or rehabilitation of the user. In the various embodiments, the communications, warnings, or messages may be prerecorded by a parent, guardian, caregiver, medical professional, or other designated user to facilitate the user in heading the communication. Messages, information, or communications from known individuals may be more impactful to the user of the smartwatch.

FIG. 14 is a flowchart of a process for categorizing a cry in accordance with an illustrative embodiment. Children and adults often cry for any number of reasons, including, but not limited to hunger, discomfort, pain, overstimulation, tiredness, boredom, injury, worry/fear, frustration, loneliness or as a condition of an illness/disease/sickness (or aforementioned issues) or so forth. Crying in infants and children is a normal, healthy means of expression and communication.

The process of FIG. 14 may be performed by utilizing the smartwatch alone or by utilizing the smartwatch and one or more associated devices. For example, a parent may user her smart phone to perform some of the associations and characterizations of FIG. 14. The process of FIG. 14 may begin by measuring characteristics of a cry of a child (step 1402). The characteristics may include volume/intensity, frequency, pattern, rhythm, breathing, duration, ability to be comforted, type of cry (e.g., shriek, sob, sputter, scream, wail, bawl, sustained cry, hysterical, etc.) and so forth. All or portions of the measurements of step 1402 may be performed automatically by the smartwatch and/or one or more associated or communicating devices. For example, a user interface of the smartwatch may allow a parent, guardian, medical professional, or other to measure characteristics of the cry. Multiple different cries may be recorded as part of the process of step 1402.

Next, the smartwatch associates the cry of the child with one or more categorizations and responses (step 1404). In one embodiment, a parent, guardian, or responsible party may associate the cry with a category or type of cry (e.g., hunger, discomfort, pain, overstimulation, tiredness, boredom, injured, as a condition of an illness/disease/sickness, frustration, loneliness, worry/fear, etc.). In one embodiment, the different categorizations and responses may have specified codes or identifiers. Custom categories may also be available through an application, programs, script, logic, or algorithm utilized by the smartwatch or an associated device. A mobile application and/or user interface may be utilized to associate the cry of the child with one or more categorizations and responses.

The response(s) may also include a treatment, actions, or efforts that were successful in helping the child stop crying, feel comforted, feel better, recover, or so forth. For example, actions may include feeding the child, holding/hugging/rocking the child, playing soothing music, playing with the child, providing a toy, giving appropriate/authorized medicine, swaddling the child, placing the child down for a nap, taking the child for medical treatment/analysis, playing video content, utilizing positive reinforcement, implementing appropriate discipline, or so forth. In one embodiment, step 1404 may be performed based on feedback from a user caring for or assigned to the child. For example, an application executed by a mobile device may be utilized to specify the categorization and response information and data. For example, a user interface may be utilized to perform the associations of the characteristics of the cry, categorizations, and one or more responses. In another embodiment, step 1404 may be performed automatically in response to biometrics, sensors, or other feedback from associated devices that determine the category and response. For example, the smartwatch or other associated devices, components, or sensors may detect that the child is being fed or listening to music.

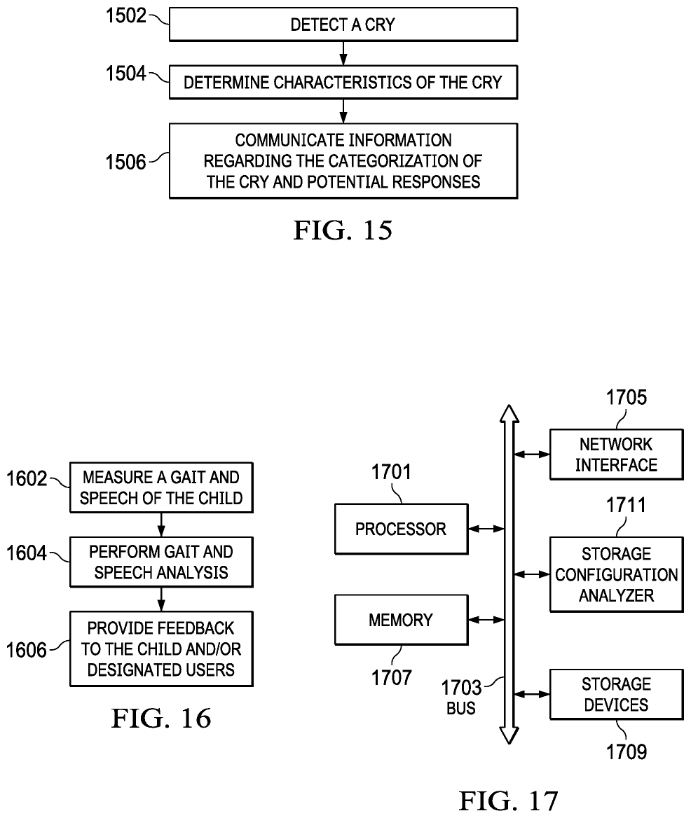

Next, the smartwatch stores the characteristics associated with the categorization and responses (step 1406). The information and data stored may be stored within the smartwatch itself, an associated device (e.g., smart phone, tablet, laptop, personal computer, etc.), servers, cloud system, or so forth. The characteristics, categorization, and responses are saved for future reference. In one embodiment, historical information may be utilized to provide information regarding the cry of the child as well as status and condition. As a result, a person helping or caring for the child may be able to quickly make determinations. Machine learning and artificial intelligence may be utilized across various populations, cohorts, groups, and so forth to provide relevant suggestions, actions, information, and data as described in FIG. 15. Data may be de-identified and shared across groups and organizations that support efforts to care for children. All communications and access may utilize security measures, protocols, and standards to apply with applicable laws, industry standards, and best practices. For example, communications may be performed by authorized users utilizing encrypted communications that comply with HIPAA.

FIG. 15 is a flowchart of a process for categorizing a cry of a child in accordance with an illustrative embodiment. The process of FIG. 15 may begin by detecting a cry (step 1502). The cry may alternatively represent one or more of a spoken/verbal outburst, non-verbal actions or communications, gestures, or so forth. For example, a microphone may measure verbal or audio produced by the child. Similarly, sensors may record biometrics of the user. Other sensors may also be utilized to measure biometrics of the child. Some children may have a non-traditional cry (e.g., non-auditory) that may require the smartwatch to detect the cry or learn based on user input (e.g., parent feedback through an associated wireless device). Besides crying, other conditions may also be recognized by the smartwatch including different emotions, status, conditions, experiences, and so forth. For example, the smartwatch may detect yelling/anger, joy/happiness/laughing, fear/nervousness, sadness/depression, surprise, and so forth.

Next, the smartwatch determines characteristics of the cry (step 1504). Characteristics of the cry may include volume/intensity, frequency, pattern, rhythm, breathing, duration, ability to be comforted, type of cry (e.g., shriek, sob, sputter, scream, wail, bawl, sustained cry, hysterical, etc.) and so forth. The smartwatch may also determine biometrics associated with the child. For example, heart rate, respiration rate, sounds/audio emissions, motion/non-motion, location, and other application information may be determined.

Next, the smartwatch communicates information regarding the categorization of the cry and potential responses (step 1506). The smartwatch may communicate information regarding the cry including a potential category and a potential response/solution/need. For example, the smartwatch may indicate that based on previous information and analysis that this “cry indicates your child is probably hungry and needs to be fed.” The smartwatch may communicate the information directly through the components and systems of the smartwatch or through an associated device. For example, a speaker of the smartwatch may announce the potential responses. One or more direct, broadcast, or networked connections may be utilized. The smartwatch may utilize historical information, time-of-day tracking, circadian rhythms and biological processes, machine learning, and user input to provide accurate categorizations and responses.

The smartwatch may also receive user input, feedback, or corrections at any time to correct errors in categorizations or responses. As a result, the smartwatch and associated system is learning, adapting, and improving at all times to provide relevant and accurate information. In one embodiment, the smartwatches may be utilized for at-risk babies in hospitals to provide information for babies/children that may be born with drug addictions. They may have a cry that may be quickly identified as withdrawal symptoms (a withdrawal cry). A “hungry” cry may also be quickly identified. The smartwatch may be utilized as part of an identification bracelet used for each baby in a hospital or other facility.

During step 1506, the smartwatch may also implement a response based on the categorization of the cry. The response may represent any number or combination of audible, visual, tactile, electrical, or other responses. For example, the smartwatch may plan an alert, message, or communication to the user/parent/guardian through the smartwatch itself. The alert may also be communicated through a network or signal to another device for playback, display, or other communication to the user. The response may also be a stimulus as previous detailed (see for example, step 1306 of FIG. 13). The response may also be a tactile response played to stop crying, prevent snoring, or stop an undesired behavior or activity. The response may also be an electrical response, such as a mild electrical shock to the arm of the user that may be utilized to reduce nausea, distract the user, or dissuade negative behavior. In other embodiments, the smartwatch may be equipped with a chemical or medical delivery system for delivering medications.

FIG. 16 is a flowchart of a process for providing feedback regarding gait and speech of a child in accordance with an illustrative embodiment. The process of FIG. 16 may be performed by the smartwatch. The process may be performed during the user's daily life and activities.

The process of FIG. 16 may begin by measuring a gait and speech of the child (step 1602). The smartwatch may utilize the various sensors to measure the movements of the child. The gait may include walking, crawling, using a wheelchair, and other motions of the child. The gait cycle of the child may include a series of rhythmical, alternative movements of the trunk and limbs resulting in the forward progress of the center of gravity of the body of the child. In one embodiment, a baseline reading for the child's gait may also be established. Readings from children from a similar cohort (e.g., age, sex, race, condition/status, etc.) may also be utilized to determine what should be a normal gait (including spatial and temporal components).

The measurements of the gait may include speed, movement (e.g., forward, lateral, sway, etc.), location, gait cycle (GC) (e.g., step length, stride length, width of walking base, foot angle, base of gait, angle of gait, stance, swing phases, etc.), timing (e.g., step time, stride time, stance time, single limb time, double limb time, swing time, cadence, speed, etc.), and so forth. The phases of the gate cycle may include the stance phase and the swing phase. The stance phase may include initial contact, loading response (flat foot), mid-stance, terminal stance (heel off), and pre-swing (toe off). The swing phase may include initial swing (acceleration), midswing (toe off), and terminal swing (deacceleration).

The speech may be measured to determine amplitude/energy, frequencies, spectral features, timing (e.g., pauses, rate, etc.), voice quality, paralinguistics (e.g., rate, speed, rhythm, inflection/vocal variety, quality, intensity, etc.) and so forth. The content of the speech (e.g., words), speed, and so forth may also be analyzed. Although described as speech, the smartwatch may also analyze and number of verbal or audio communications (e.g., crying, grunting, yelling, screaming, whining, fussing, hissing, gutturalizations, humming, groaning, laughing, giggling, panting, sighing, whispering, etc.). The speech may also represent any number verbalizations or audible emissions. The smartwatch (and associated devices/systems) may also measure gestures, non-verbal communications (e.g., sign language), body language, proxemics, haptics, facial expressions, motions associated with communications or reactions, and so forth.

Next, the smartwatch performs gait and speech analysis (step 1604). In one embodiment, the gait and speech analysis may be performed by the smartwatch. In another embodiment, the data and information associated with the gait and speech of the child may be offloaded to a smart phone, home hub, personal computer, cloud system, or so forth for detailed analysis. For example, the gait and speech data may be offloaded for analysis using a smart phone executing an application specifically configured for receiving the measurements from step 1602 and performing analysis of the measurements to determine relevant information. Raw data or processed data may be offloaded for additional analysis. Thus, some processing may be performed by the smartwatch before communication. The gait analysis may measure the gait cycle of the child including the various phases, spatial and temporal parameters of the gait cycle. Various types of kinematic and kinetic analysis may be performed for the various measurements and variables (e.g., distance, time, joint angles, body angles, gait analysis). The kinematic gait analysis may include the description of gait components. The kinematic gait analysis includes the temporal and spatial variables of the gait cycle.

The speech may be analyzed to determine the various factors described above. The speech of the child may be analyzed to determine the status of the child. For example, is the child distressed, happy, sad, talking, silent, angry, progressing, or so forth.

Step 1604 may be utilized to determine whether the child is progressing in walking/crawling and speaking. For example, the smartwatches may be assigned by hospitals, special needs schools, facilities, treatment areas, or so forth. Any number of rating systems, categorizations, scores, recommendations, suggestions, grades, or other information may be assigned to the gait and speech of the child.

Next, the smartwatch provides feedback to the child and/or designated users (step 1606). The measurements, information, and analysis gathered during the process of FIG. 16 may be communicated to the user/wearer of the smartwatch (e.g., child user), parents/guardians, medical professionals, administrators, or others. The feedback may be provided audibly through a speaker of the smartwatch, utilizing one or more displays/lights, utilizing tactile feedback (e.g., vibration components, haptics, etc.), electrical currents/impulses, or so forth. The feedback may also be communicated to an associated device, such as a smart phone, tablet, wireless speaker, laptop, personal computer, or so forth. In one embodiment, the feedback may be encouragement such as, “stand up and try to walk again” or “you can crawl a little more” or “please wheel yourself forward ten more feet.” The feedback may also include commands or instructions, such as “keep your stance a little wider”, “walk with your head up”, “remember your posture”, or other applicable information. During step 1606, specific guidance may be given for the child or someone nearby facilitating the child. For example, the guidance may be as relevant to a parent helping the child as it is to the child itself. The smartwatch may also provide guidance regarding areas that are safe or unsafe for walking or mobility. For example, the child may be instructed to always use the handrail when walking stairs, avoid roads/streets, cars, and walking with a parent. The instructions or feedback may be prerecorded by a parent, sibling, family member, guardian, trainer, teacher, or other individual associated with the user.

The illustrative embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments of the inventive subject matter may take the form of a computer program product embodied in any tangible medium of expression having computer usable program code embodied in the medium. The described embodiments may be provided as a computer program product, or software, that may include a machine-readable medium having stored thereon instructions, which may be used to program a computing system (or other electronic device(s)) to perform a process according to embodiments, whether presently described or not, since every conceivable variation is not enumerated herein. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions. In addition, embodiments may be embodied in an electrical, optical, acoustical or other form of propagated signal (e.g., carrier waves, infrared signals, digital signals, etc.), or wireline, wireless, or another communications medium.

Computer program code for carrying out operations of the embodiments may be written in any combination of one or more programming languages, including an object-oriented programming language such as Java, Smalltalk, C++ or the like and conventional procedural programming languages, such as the “C” programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN), a personal area network (PAN), or a wide area network (WAN), or the connection may be made to an external computer (e.g., through the Internet using an Internet Service Provider).

FIG. 17 depicts a computing system 1700 in accordance with an illustrative embodiment. For example, the computing system 1700 may represent a device, such as one or more of the devices 101 of FIG. 1. The computing system 1700 includes a processor unit 1701 (possibly including multiple processors, multiple cores, multiple nodes, and/or implementing multi-threading, etc.). The computing system includes memory 1707. The memory 1707 may be system memory (e.g., one or more of cache, SRAM, DRAM, zero capacitor RAM, Twin Transistor RAM, eDRAM, EDO RAM, DDR RAM, EEPROM, NRAM, RRAM, SONOS, PRAM, etc.) or any one or more of the above already described possible realizations of machine-readable media. The computing system also includes a bus 1703 (e.g., PCI, ISA, PCI-Express, HyperTransport®, InfiniBand®, NuBus, etc.), a network interface 1705 (e.g., an ATM interface, an Ethernet interface, a Frame Relay interface, SONET interface, wireless interface, etc.), and a storage device(s) 1709 (e.g., optical storage, magnetic storage, etc.). The system memory 1707 embodies functionality to implement embodiments described above. The system memory 1707 may include one or more functionalities that store personal data, parameters, application, user profiles, and so forth. Code may be implemented in any of the other devices of the computing system 1700. Any one of these functionalities may be partially (or entirely) implemented in hardware and/or on the processing unit 1701. For example, the functionality may be implemented with an application specific integrated circuit, in logic implemented in the processing unit 1701, in a co-processor on a peripheral device or card, etc. Further, realizations may include fewer or additional components not illustrated in FIG. 17 (e.g., video cards, audio cards, additional network interfaces, peripheral devices, etc.). The processor unit 1701, the storage device(s) 1709, and the network interface 1705 are coupled to the bus 1703. Although illustrated as being coupled to the bus 1703, the memory 1707 may be coupled to the processor unit 1701.

The features, steps, and components of the illustrative embodiments may be combined in any number of ways and are not limited specifically to those described. In particular, the illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A method for monitoring a child utilizing a smart wearable, comprising:
measuring biometrics of the child utilizing at least the smart wearable;
monitoring a status of the child and of the smart wearable;
generating an alert in response to any of determining the infant has had an impact greater than a threshold, moves beyond a geo-fenced area, one or more of the biometrics exceeds a threshold, and determining the smart wearable has been cut, damaged, or broken.

2. The method of claim 1, wherein the alert is sent in response to a band of the smartwatch being cut or removed from an arm of the child without authorization.

3. The method of claim 1, wherein the smart wearable is a smart watch worn by the child, and where the

4. The method of claim 1, further comprising:
communicating the biometrics of the child to one or more specified devices at specified intervals.

5. The method of claim 1, wherein the smart wearable communicates with a plurality of devices to determine the location of the child and movements of the child.

6. A method for monitoring a user, comprising:
determining activities of a user utilizing a smartwatch;
automatically determining whether a stimuli is appropriate; and
providing stimuli to the user utilizing the smartwatch in response to determining the stimuli are appropriate.

7. The method of claim 6, wherein the stimuli is one or more of content displayed by a display of the smartwatch, audio played by one or more speakers of the smart watch, tactile output from the smartwatch, and electrical impulses applied through contacts of the smartwatch.

8. The method of claim 256, further comprising:
measuring biometric data of the user, wherein the biometric data is utilized to determine whether the stimuli is appropriate.

9. The method of claim 6, wherein the stimuli is a video, game, or a song.

10. The method of claim 6, further comprising:
establishing user preferences for determine when the stimuli is appropriate.

11. The method of claim 6, wherein the stimuli include one or more stimulus that are preestablished by a parent, guardian, caregiver, or medical professional.

12. A method for providing information regarding a user, comprising:
detecting a cry of a user wearing a smartwatch;
determining characteristics of the cry detected by the smartwatch; and
communicating information regarding an associated categorization of the cry and potential responses to the cry of the user.

13. The method of claim 12, further comprising:
measuring characteristics of a cry of a user;
associating the cry of the user with one or more categorizations and responses; and
storing the characteristics with the categorization and responses.

14. The method of claim 12, wherein the characteristics include at least amplitude, frequency, timing, and voice quality.

15. The method of claim 12, wherein the categorization indicates a potential status or condition of the user.

16. The method of claim 12, wherein the user is a child.

17. The method of claim 12, wherein the responses are selected from holding the user, comforting the user, feeding the user, putting the user down for bed, entertaining the user, and providing medicine or treatment to the user.

18. A method for providing feedback to a user, comprising:
measuring a gait and speech of a user utilizing a smartwatch worn by the user;
performing gait and speech analysis of the user; and
providing feedback to the user and/or designated users regarding the gait and speech of the user.

19. The method of claim 18, further comprising:
establishing baseline readings for the gait and speech of the user.

20. The method of claim 18, wherein the user is a child or a person going through rehabilitation.

21. The method of claim 18, wherein the gait analysis includes determining speed, location, gait cycle, and timing.
